(19) United States
(12) Patent Application Publication
(10) Pub. No.: US 2025/0197842 A1
(43) Pub. Date: Jun. 19, 2025

(54) IMPROVING ENZYMATIC CHANNELING EFFICIENCY VIA MIXED NANOPARTICLE SCAFFOLDS WITHIN SELF-ASSEMBLED NANOPARTICLE ENZYME CLUSTERS

(71) Applicant: The Government of the United States of America, as represented by the Secretary of the Navy, Arlington, VA (US)

(72) Inventors: Igor L. Medintz, West Springfield, VA (US); Shelby L. Hooe, Lorton, VA (US); Christopher M. Green, Lorton, VA (US); Kimihiro Susumu, Alexandria, VA (US); Michael H. Stewart, Springfield, VA (US)

(73) Assignee: The Government of the United States of America, as represented by the Secretary of the Navy, Arlington, VA (US)

(21) Appl. No.: 18/981,084

(22) Filed: Dec. 13, 2024

Related U.S. Application Data
(60) Provisional application No. 63/609,397, filed on Dec. 13, 2023.

(51) Int. Cl. C12N 11/14; B82Y 30/00; C12N 9/02; C12N 9/04; C12N 9/12; C12N 9/88; C12N 9/92; C12N 11/18

(52) U.S. Cl. CPC C12N 11/14; C12N 9/0006; C12N 9/0008; C12N 9/1205; C12N 9/88; C12N 9/92; C12N 11/18; B82Y 30/00; C12Y 101/01026; C12Y 102/01012; C12Y 207/01002; C12Y 207/01011; C12Y 207/0104; C12Y 207/02003; C12Y 401/02013; C12Y 503/01001; C12Y 503/01009

(57) ABSTRACT

Quantum dots (QDs) and nanoplatelets (NPLs) are two types of nanoparticles used as scaffolds for enzymes operating in enzymatic cascades. Combinations of QDs and NPLs were surprisingly found to operate synergistically to create a greater enhancement than either alone when operating as scaffolds for enzymatic cascade reactions. A process involves providing an enzymatic cascade including a cluster of nanoparticles including both QDs and NPLs and having a plurality of enzymes bound thereto, the enzymes configured as an enzymatic cascade, such that the product of a first enzyme is a substrate of a second enzyme; contacting the cascade cluster with a substrate of the first enzyme; and allowing a reaction to proceed so that each of the plurality of enzymes acts in succession to produce an end product. The enzymes are bound to the nanoparticles via metal affinity coordination between histidine tags on the enzymes and zinc-containing surfaces of the nanoparticles.

### CROSS-REFERENCE TO RELATED APPLICATIONS

This application claims priority to U.S. Provisional Patent Application No. 63/609,397 filed on Dec. 13, 2023, and is further related to U.S. Pat. Nos. 11,512,305 and 11,795,483; U.S. Patent Application Publication No. U.S. 2022/0307004; and U.S. Provisional Patent Application Nos. 63/482,369 filed Jan. 31, 2023, 63/503,983 filed May 24, 2023, and 63/507,556 filed Jun. 12, 2023, the complete disclosures of which, in their entireties, are incorporated by reference herein.

### FEDERALLY-SPONSORED RESEARCH AND DEVELOPMENT

The United States Government has ownership rights in this invention. Licensing inquiries may be directed to Office of Technology Transfer, US Naval Research Laboratory, Code 1004, Washington, DC 20375, USA; +1.202.767.7230; techtran@nrl.navy.mil, referencing NC 211659.

### INCORPORATION BY REFERENCE

This application incorporates by reference the Sequence Listing XML file submitted via the patent office electronic filing system having the file name “NC211659-US2.xml” and created on Feb. 25, 2025, with a file size of 4 kilobytes.

### BACKGROUND

### Field of the Invention

The embodiments herein generally relate to enzymatic cascade reactions and bio-nanotechnology, and more particularly to methods and compositions for enhancing enzymatic cascade reactions using mixed nanoparticle scaffolds comprising quantum dots and nanoplatelets for achieving enhanced substrate channeling and improved product formation in cell-free synthetic biology applications.

### Background of the Invention

This background description includes information that may be useful in understanding the present invention. It is not an admission that any of the information provided herein is prior art or relevant to the presently claimed invention or that any publication specifically or implicitly referenced is prior art.

The ability to synthesize both simple and complex molecules via green, sustainable synthetic strategies continues to drive current research interest in the field of synthetic biology. Cell-based approaches to synthetic biology represent a robust and cost efficient route to synthesize desired molecules on an industrial scale because of the ability to use large fermenters filled with self-replicating, active cellular cultures. However, within cell-based systems the full synthetic potential of a given enzyme and/or multi-enzyme cascade cannot be realized. This is because within a cellular system the production of the enzyme(s) cannot exceed that of the cell's tolerance and resistance to toxicity. Therefore, one such viable alternative approach to overcome the limits imposed by cellular toxicity is the application of minimalist cell-free synthetic biology. This type of cell-free biosynthesis needs only the enzyme(s), substrate(s), cofactor(s), and buffer required for the formation of a desired product. Minimalist cell-free synthetic biology is an appealing approach when attempting to implement non-natural or xenobiotic substrates, but it is also an advantageous strategy for optimizing a single target pathway where the concentrations/ratios of enzyme(s), substrate(s), and cofactor(s) can be directly controlled and modified to enhance the formation of a desired product. One apparent challenge associated with minimalist cell-free synthetic biology is the lack of channeling that nature provides within cellular systems, whereby the confines of the cell facilitate efficient catalysis by localizing enzymes and reducing native substrate diffusion through the cellular membrane. There exists a variety of materials, including DNA-, MOF-, and nanoparticle-based strategies, that have been developed to localize enzymes in close proximity to one another such that conditions for channeling can be accessed. Further, these material-based immobilization strategies can also function to stabilize enzymes and enhance the individual activity of a given enzyme.

One significant challenge in cell-free synthetic biology is achieving the enhanced catalytic efficiency found in natural cellular systems. Within cells, enzymes often form dense clusters or metabolons through transient interactions that facilitate channeling—the most efficient form of multi-enzyme catalysis. Channeling phenomena arise when at least two enzymes are physically held in close proximity such that intermediates formed by one enzyme reach the proceeding enzyme faster than they can diffuse into bulk solution. As a nanoscale phenomenon, channeling is observable under diffusion-limited reaction conditions when the effective multienzyme catalytic rate exceeds the diffusion rate of intermediates away from the enzyme complex.

While fusion of enzymes or attachment to organic scaffolds has been attempted to achieve channeling, these approaches have not proven consistently reliable. An alternative approach utilizes nanoparticles as inorganic scaffolds that crosslink enzymes into dense clusters where probabilistic channeling can occur. Previous work has shown that both quantum dots (QDs) and nanoplatelets (NPLs) can individually enhance enzymatic cascade reactions through this mechanism. However, as described herein, the combination of different types of nanoparticles in defined ratios provides unexpected synergistic improvements in reaction efficiency beyond what either type alone can achieve.

The self-assembly of these mixed nanoparticle-enzyme systems follows a diffusion-limited aggregation (DLA) mechanism. In this process, particles undergoing Brownian motion follow random walk paths and cluster together upon interaction. For the systems described herein, both the nanoparticles and multimeric enzymes participate in this assembly process through metal affinity coordination between histidine tags on the enzymes and the zinc-containing nanoparticle surfaces. The resulting clusters provide the high local enzyme density necessary for efficient substrate channeling while maintaining colloidal stability.

As previously reported, the use of QDs and NPLs were each individually found to enhance enzymatic cascade reactions; i.e., those wherein the product of a first enzyme is the substrate of a second enzyme and so forth (see U.S. Pat. Nos. 11,512,305 and 11,795,483, respectively). For example, QD immobilization of pyruvate kinase PykA and lactate dehydrogenase (LDH) for the two-enzyme conversion of pyruvic acid to lactic acid has resulted in a 100-fold improvement in product formation due to channeling. Notably, in this system the quaternary structure of the LDH enzyme was significantly stabilized by QD immobilization which resulted in improved activity at lower enzyme concentrations. The first enzyme in the cascade is pyruvate kinase (PykA, EC 2.7.1.40) which converts phosphoenolpyruvate (PEP) to pyruvic acid using the adenosine diphosphate (ADP) as the phosphate acceptor to form adenosine triphosphate (ATP). The PykA gene encodes a 53.5 kDa monomer which assembles into the final active 220 kDa homotetramer. The second enzyme in the cascade is lactate dehydrogenase (LDH, EC 1.1.1.28) which converts pyruvic acid to lactic acid using nicotinamide adenine dinucleotide (NADH) as the reducing cofactor. The LDH gene encodes a 39.1 kDa monomer which assembles into the final active 160 kDa homotetramer.

Intermediary or probabilistic channeling in multi-enzyme cascades represents a powerful tool to achieve environmentally sustainable, one-pot synthetic strategies. In particular, multi-enzyme cascades in vitro offer greater synthetic versatility due to the ability to both mix purified enzymes from different sources and bypass cellular toxicity. A need exists for improvements in such techniques.

Accordingly, there remains an unmet need for improving the efficiency and stability of enzyme cascade reactions in cell-free systems while maintaining the ability to work with both natural and non-natural substrates. Prior attempts using single types of nanoparticle scaffolds have shown limited success in achieving optimal enzyme proximity and activity.

### SUMMARY

As described herein, combinations of QDs and NPLs were surprisingly found to operate synergistically to create a greater enhancement than either alone when operating as scaffolds for enzymatic cascade reactions.

In one embodiment, a method of conducting a cascade reaction includes providing an enzymatic cascade comprising a cluster of nanoparticles having a plurality of enzymes bound thereto, the plurality of enzymes configured as an enzymatic cascade wherein the product of a first enzyme is the substrate of a second enzyme and so forth; contacting the cascade cluster with a substrate of the first enzyme; and allowing a reaction to proceed so that each of the plurality of enzymes acts in succession to produce an end product, wherein the cluster of nanoparticles comprises both QDs and NPLs and wherein the plurality of enzymes comprises (a) pyruvate kinase PykA and lactate dehydrogenase, or (b) glucokinase, phosphoglucose isomerase, phosphofructokinase, aldolase, triose phosphate isomerase, glyceraldehyde 3-phosphate dehydrogenase, and phosphoglycerate kinase.

In another embodiment, an enzymatic cluster comprises cluster of nanoparticles having a plurality of enzymes bound thereto, the plurality of enzymes configured as an enzymatic cascade; wherein the cluster of nanoparticles comprises both QDs and NPLs; and wherein the plurality of enzymes comprises (a) pyruvate kinase PykA and lactate dehydrogenase, or (b) glucokinase, phosphoglucose isomerase, phosphofructokinase, aldolase, triose phosphate isomerase, glyceraldehyde 3-phosphate dehydrogenase, and phosphoglycerate kinase.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings. The patent or application file contains at least one drawing executed in color. Copies of this patent or patent application publication with color drawing(s) will be provided by the Office upon request and payment of the necessary fee.

FIG. 1A provides a schematic illustrating different strategies examined for the possibility of improved intermediary channeling, according to an embodiment herein.

FIG. 1B depicts the enzyme pathway utilized to convert phosphoenolpyruvic acid to lactic acid, according to an embodiment herein.

FIGS. 1C-1F provide transmission electron micrograph (TEM) images of the ca. 525, 625, and 641 nm emitting CdSe/CdS/ZnS core/shell/shell QDs, and NPLs, respectively, according to the embodiments herein.

FIG. 2A illustrates mixed nanoparticle-enzyme clusters formed with the QDs and NPLs, according to an embodiment herein.

FIG. 2B is a representative TEM micrograph from a sample mixture containing nanoclusters, according to an embodiment herein.

FIGS. 3A-3F show the kinetic enhancement from channeling in the two-enzyme cascade across different individual nanoparticles, according to the embodiments herein.

FIGS. 4A-4D show the changes in kflux in the two-enzyme cascade as the result of mixed QD-NPL clusters engaged in channeling, according to the embodiments herein.

FIGS. 5A-5F show the changes in kflux in the two-enzyme cascade as the result of mixed QDs of different sizes, according to the embodiments herein.

FIGS. 6A-6D show the changes in kflux in the two-enzyme cascade from mixing QDs with NPLs or other QDs at a constant overall concentration, according to the embodiments herein.

FIGS. 7A-7C show the changes in kflux in a seven-enzyme cascade from mixing QDs with NPLs at a constant overall nanoparticle concentration, according to the embodiments herein.

FIG. 8 is a block diagram illustrating an enzymatic cluster composition, according to an embodiment herein.

FIG. 9 is a flow diagram illustrating a method of conducting a cascade reaction, according to an embodiment herein.

FIG. 10 is a flow diagram illustrating a method of enhancing enzymatic cascade reactions, according to an embodiment herein.

### DETAILED DESCRIPTION

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein. The following description of particular embodiment(s) is merely exemplary in nature and is in no way intended to limit the scope of the invention, its application, or uses, which can, of course, vary.

It will be understood that when an element or layer is referred to as being “on”, “connected to”, or “coupled to” another element or layer, it may be directly on, directly connected to, or directly coupled to the other element or layer, or intervening elements or layers may be present. In contrast, when an element or layer is referred to as being “directly on”, “directly connected to”, or “directly coupled to” another element or layer, there are no intervening elements or layers present. It will be understood that for the purposes of this disclosure, “at least one of X, Y, and Z” or “any of X, Y, and Z” may be construed as X only, Y only, Z only, or any combination of two or more items X, Y, and Z (e.g., XYZ, XY, XZ, YZ).

The description herein describes inventive examples to enable those skilled in the art to practice the embodiments herein and illustrates the best mode of practicing the embodiments herein. Upon reading the following description in light of the accompanying drawing figures, those skilled in the art will understand the concepts of the disclosure and will recognize applications of these concepts not particularly addressed herein.

The terms first, second, etc. may be used herein to describe various elements, but these elements should not be limited by these terms as such terms are only used to distinguish one element from another. For example, a first element could be termed a second element, and, similarly, a second element could be termed a first element, etc. without departing from the scope of the present disclosure. As used herein, the term “and/or” includes any and all combinations of one or more of the associated listed items.

Furthermore, although the terms “final”, “first”, “second”, “upper”, “lower”, “bottom”, “side”, “intermediate”, “middle”, and “top”, etc. may be used herein to describe various elements, but these elements should not be limited by these terms. These terms are only used to distinguish one element from another. For example, a first element could be termed an “top” element and, similarly, a second element could be termed a “top” element depending on the relative orientations of these elements.

The terminology used herein is for the purpose of describing particular embodiments only and is not intended to be limiting of the disclosure. As used herein, the singular forms “a”, “an”, and “the” are intended to include the plural forms as well, unless the context clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used herein, specify the presence of stated features, regions, integers, steps, operations, elements, and/or components, but do not preclude the presence or addition of one or more other features, regions, integers, steps, operations, elements, components, and/or groups thereof. The term “or a combination thereof” means a combination including at least one of the foregoing elements.

Unless otherwise defined, all terms (including technical and scientific terms) used herein have the same meaning as commonly understood by one of ordinary skill in the art to which this disclosure belongs. It will be further understood that terms such as those defined in commonly used dictionaries, should be interpreted as having a meaning that is consistent with their meaning in the context of the relevant art and the present disclosure, and will not be interpreted in an idealized or overly formal sense unless expressly so defined herein.

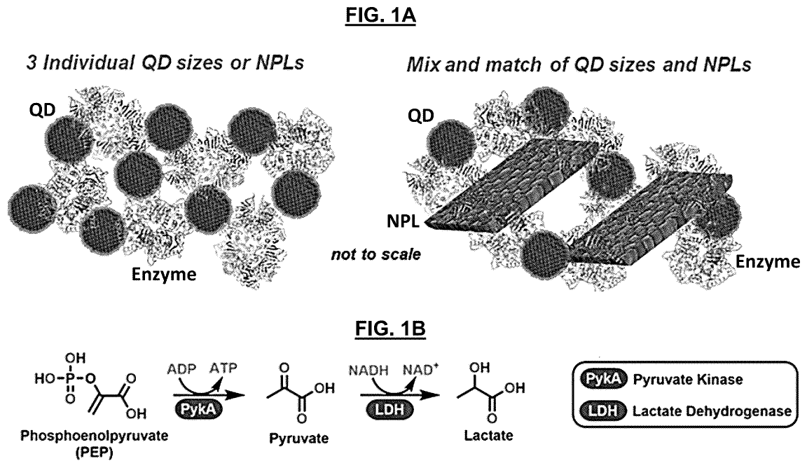

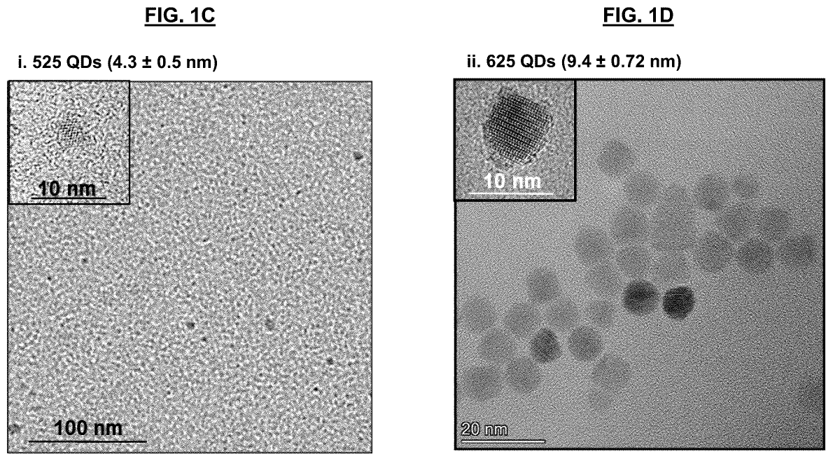

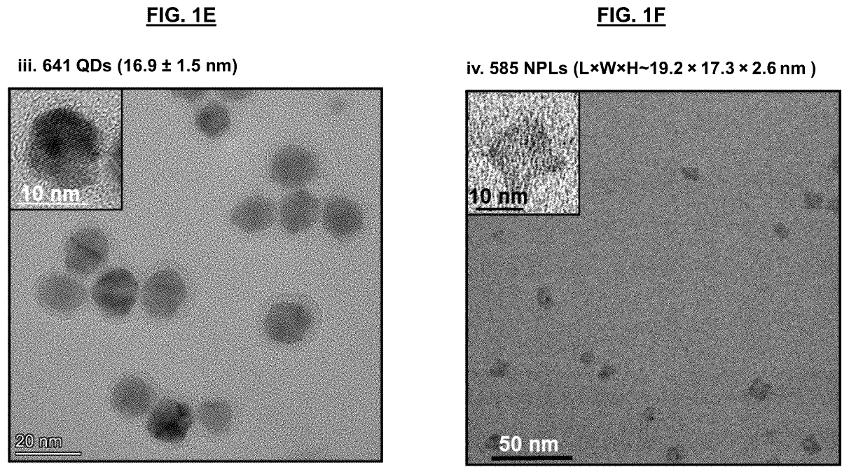

Referring now to the drawings, and more particularly to FIGS. 1A through 10, where similar reference characters denote corresponding features consistently throughout, there are shown exemplary embodiments. In the drawings, the size and relative sizes of components, layers, and regions, etc. may be exaggerated for clarity.

### Definitions

Before describing the present invention in detail, it is to be understood that the terminology used in the specification is for the purpose of describing particular embodiments, and is not necessarily intended to be limiting. Although many methods, structures and materials similar, modified, or equivalent to those described herein can be used in the practice of the present invention without undue experimentation, the preferred methods, structures and materials are described herein. In describing and claiming the present invention, the following terminology will be used in accordance with the definitions set out below.

As used herein and as mentioned above, the singular forms “a”, “an,” and “the” do not preclude plural referents, unless the content clearly dictates otherwise.

As used herein and as mentioned above, the term “and/or” includes any and all combinations of one or more of the associated listed items.

As used herein, the term “about” when used in conjunction with a stated numerical value or range denotes somewhat more or somewhat less than the stated value or range, to within a range of ±10% of that stated.

The terms “semiconductor nanocrystal,” “quantum dot,” and “QD” are used interchangeably herein and refer to an inorganic crystallite of about 1 nm or more and about 1000 nm or less in diameter or any integer or fraction of an integer therebetween, preferably at least about 2 nm and about 50 nm or less in diameter or any integer or fraction of an integer therebetween, more preferably at least about 2 nm and about 20 nm or less in diameter (for example about 2, 3, 4, 5, 6, 7, 8, 9, 10, 11, 12, 13, 14, 15, 16, 17, 18, 19, or 20 nm). QDs are characterized by their relatively uniform nanometer size. A QD is capable of emitting electromagnetic radiation upon excitation (the QD is luminescent) and includes a “core” of one or more first semiconductor materials, with the core optionally surrounded by a “shell” of a second semiconductor material.

The term “nanoparticle” or “NP” as used herein includes the above-mentioned QDs, the below-mentioned nanoplatelets, and other nano-scale and smaller particles such as metallic nanoparticles (e.g., nanoparticles comprising Ag, Au, Cu, Pd, Pt, and combinations thereof), carbon nanotubes, proteins, polymers, dendrimers, viruses, and drugs. A nanoparticle has a size of less than about 1 micron, optionally less than about 900, 800, 700, 600, 500, 400, 300, 200, 100, 80, 60, 50, 40, 30, 20, 10, 9, 8, 7, 6, 5, 4, 3, 2, or 1 nanometers. A nanoparticle may have various shapes such as a rod, a tube, a sphere, and the like. Nanoparticles may be made from various materials including metals, carbon (such as carbon nanotubes), polymers, and combinations thereof.

The term “nanoplatelet” or “NPL” refers to a nanoparticle with a non-spherical shape, including, for example, an oblate spheroid shape, or a quasi-two-dimensional and roughly circularly shape. In embodiments, NPLs have lateral dimensions from tens to hundreds of nanometers, and thicknesses of about 1 to 3 nm.

The term “nanomaterial” refers to a material having at least one dimension of less than 100 nm and that is not a naturally-occurring material. NPs and NPLs are normally considered nanomaterials.

The term “channeling” refers to the process where reaction intermediates are directly transferred between sequential enzymes without diffusing into bulk solution.

The term “Metabolon” refers to a temporary structural-functional complex formed between sequential enzymes in a metabolic pathway.

The terms “Coupled catalytic flux” or “kflux” refer to the rate at which substrates are processed through multiple sequential enzyme reactions in a cascade.

The term “Diffusion-limited aggregation” or “DLA” refers to the process by which particles undergoing Brownian motion cluster together upon random collisions.

### Overview

Combinations of QDs with the NPLs in a mixed-assembly system were examined and found to achieve a greater enhancement relative to the free enzymes than can be obtained from any of the single nanomaterial assemblies.

The synergistic enhancement observed with mixed QD-NPL systems was unexpected, as previous work with individual nanoparticle types suggested that geometric complementarity between different scaffold materials would lead to decreased stability and increased aggregation.

Using a two-enzyme cascade comprising of pyruvate kinase PykA and lactate dehydrogenase (LDH) for the conversion of phosphoenolpyruvic acid to lactic acid in conjunction with semiconductor QDs as a prototypical system, the formation of enzyme clusters dictated overall rate of coupled catalytic flux (k_{flux}) across a series of differentially sized and shaped nanomaterials ranging from 2D NPLs to QDs of varying size. By mixing different nanoparticulate materials into these self-assembled clusters, a >10× improvement in k_{flux} was observed relative to the free enzymes, which is also 2× greater or more than that of the enhancement achieved on individual NPs. Cluster formation was confirmed and characterized with gel electrophoresis and TEM imaging. The generalizability of this mixed NP approach to improving flux in the channeled nanoclusters is confirmed by initial application to a more complex seven-enzyme system. Overall, this represents a powerful approach for accessing channeling phenomena with almost any choice of enzymes constituting a multienzyme cascade.

The enhanced performance of mixed QD-NPL systems stems from several complementary mechanisms. The different geometries of the nanoparticles—spherical QDs and planar NPLs—create unique spatial arrangements that optimize enzyme positioning. This geometric complementarity allows for higher local enzyme densities while maintaining colloidal stability. The NPLs provide extended flat surfaces for enzyme attachment, while the smaller QDs can fill interstitial spaces and create additional crosslinking points. This arrangement leads to more stable quaternary structures of the enzymes and improved substrate channeling efficiency.

The mixed system also provides enhanced control over cluster size and morphology. The NPLs act as organizing centers that can accommodate multiple enzyme attachments across their surface, while the QDs provide additional attachment points that help prevent excessive aggregation. This balanced assembly approach results in clusters that maintain optimal size for channeling while avoiding the precipitation issues that can occur with NPL-only systems at higher concentrations.

Furthermore, the mixed systems demonstrate improved tolerance to varying reaction conditions. When environmental parameters such as pH, temperature, or ionic strength fluctuate, the presence of both types of nanoparticles provides greater structural stability to the enzyme clusters. This enhanced stability translates to more consistent catalytic performance across a broader range of conditions.

This detailed analysis examines the two-enzyme cascade comprising of PykA and LDH for the conversion of phosphoenolpyruvic acid to lactic acid, with particular focus on the novel application of mixed nanoparticle systems combining QDs of varying size with NPLs. The goal was to identify optimal conditions for enhancing product formation through careful optimization of the underlying cluster architecture (FIGS. 1A and 1B). The insights gained from this two-enzyme system were then successfully applied to a more complex seven-enzyme system, demonstrating the broader applicability of this approach.

The development of these mixed nanoparticle systems represents a significant advance in cell-free synthetic biology. By providing enhanced control over enzyme organization and improving reaction efficiency, this approach opens new possibilities for the synthesis of both natural and non-natural products. The ability to tune system parameters through nanoparticle selection and ratio optimization offers unprecedented flexibility in designing efficient enzymatic cascade reactions.

### EXAMPLES

The first enzyme in the two-enzyme cascade is PykA (EC 2.7.1.40), which converts phosphoenolpyruvate (PEP) to pyruvic acid using adenosine diphosphate (ADP) as the phosphate acceptor to form adenosine triphosphate (ATP). The PykA gene encodes a ˜53.5 kDa monomer, which assembles into the final active ˜220 kDa homotetramer. The second enzyme in the cascade is LDH (EC 1.1.1.28) which converts pyruvic acid to lactic acid using nicotinamide adenine dinucleotide (NADH) as the reducing cofactor (FIG. 1B). The LDH gene encodes a ˜39.1 kDa monomer, which assembles into the final active ˜160 kDa homotetramer. Both enzymes were cloned directly from E. coli strain BL21(DE3) and inserted into the pET28b vector and are expressed with an N-terminal (His)₆ tag on their respective monomers. Coupled PykA-LDH activity functions in downstream glycolysis as part of glucose metabolism to energy by regenerating ATP.

The examined group of NPs included spherical ca. 525 nm emitting (average diameter˜4.3±0.5 nm), 625 nm emitting (average diameter˜9.4±0.72 nm), and 641 nm emitting (average diameter˜16.9±1.5 nm) CdSe/CdS/ZnS core/shell/shell QDs and ˜585 nm emitting CdSe/ZnS core/shell NPLs (four monolayers CdSe) with an average L×W×H of ˜19.2×17.3×2.6 nm, as seen in FIG. 1C. The latter are a quasi-two-dimensional QD-like material. As noted above in the Definitions section, NP or nanomaterial is used interchangeable to refer generically to these materials while QDs and NPLs are used to specify a given type. The NP materials were surface functionalized with the zwitterionic dihydrolipoic acid derivative compact ligand 4 (CL4, see U.S. Pat. No. 8,859,284), which replace the hydrophobic ligands utilized during nanomaterial crystal growth for colloidal stability in aqueous buffers. For enzyme attachment or bioconjugation to the NPs, self-assembly was driven by metal affinity coordination of the enzyme's pendant (His)₆-motifs to the QD's ZnS shell. This well-known cooperative, high-affinity, interaction (K_{d}˜1 nM) occurs almost spontaneously and follows a Poisson distribution mechanism with the upper packing limit of a monomeric protein around a given NP dictated by the geometric fitting constraints of that protein based on size and shape. The (His)₆-tags bind at available ZnS sites on the NP's surface and do not displace the already coordinated CL4 ligands.

FIGS. 1A-1B illustrate an overview of the two-enzyme channeled system converting phosphoenolpyruvate to lactate. FIG. 1A is a schematic diagram illustrating the two different strategies utilized to access and improve intermediary channeling, according to the embodiments herein. FIG. 1B depicts the enzyme pathway utilized according to the embodiments herein to convert phosphoenolpyruvic acid to lactic acid, → indicates enzymatically catalyzed step(s). Chemical structures of the substrate, intermediaries, and final product are shown for the two-enzyme pathway containing PykA and LDH. This is referred to as the ‘two-enzyme pathway’ generically to distinguish it from another enzyme cascade utilized later. FIGS. 1C-1F are representative TEMs of the ca. (i) 525, (ii) 625, (iii) 641 nm emitting CdSe/CdS/ZnS core/shell/shell QDs, and (iv) NPLs used according to the embodiments herein. The inset shows a high-resolution image of each material. The well-dispersed and non-aggregated appearance of the QDs in the absence of enzyme can be seen. The small size of the 525 nm emitting QDs approaches the limit of resolution of the TEM.

The QDs utilized comprise CdSe/CdS/ZnS core/shell/shell structures with carefully controlled size distributions: 525 nm emitting QDs (average diameter 4.3±0.5 nm), 625 nm emitting QDs (average diameter 9.4±0.7 nm), and 641 nm emitting QDs (average diameter 16.9±1.5 nm). The nanoplatelets comprise CdSe/ZnS core/shell structures (four monolayers CdSe) with average dimensions of 19.2×17.3×2.6 nm and emission at approximately 585 nm.

Both types of nanoparticles are surface functionalized with the zwitterionic dihydrolipoic acid derivative compact ligand 4 (CL4). The CL4 ligand is synthesized by first dissolving the disulfide, methyl ester form (126 mg, 0.3 mmol) in ethanol (1 mL) and DI water (0.5 mL) followed by stirring with LiOH (16 mg, 0.67 mmol). After pH adjustment to 7-8, reduction with NaBH4 (25 mg, 0.66 mmol) provides the active form. This surface modification replaces the hydrophobic ligands used during nanoparticle synthesis and provides excellent colloidal stability in aqueous buffers while still allowing metal affinity coordination of enzymes.

The CL4 ligand synthesis proceeds with clear visual indicators of reaction progress. After addition of NaBH4, the solution is stirred until it turns completely colorless, indicating successful reduction. The activated ligand solution is then combined with CdSe/ZnS NPLs (2 nmol) in toluene that have been precipitated with minimal isopropanol and centrifuged at 3500 rpm for 5 minutes. The NPLs are dissolved in 1 mL of chloroform and added to the activated ligand mixture with vigorous stirring. Small portions of chloroform and DI water are added until a biphasic mixture is achieved. The mixture is rapidly stirred until the NPLs transfer to the aqueous phase (2-24 hours). The organic phase is discarded, and the aqueous phase is washed with CHCl₃ (3×1 mL). The aqueous phase is filtered through a 0.45 m hydrophilic membrane filter and washed with DI water (2-3×1.5 mL) using a centrifugal filtration device (e.g., Millipore®, MW cutoff 100 kDa). The final aqueous CL4-capped NPLs are stored at 4° C. in the dark until use.

The enzymes are expressed with N-terminal hexahistidine (His6) tags using the following sequences:

Pyruvate kinase PykA (SEQ ID NO 1):
MGSSHHHHHHSSGLVPRGSHMSRRLRRTKIVTTLGPATDRDNNLEKVIAA
GANVVRMNFSHGSPEDHKMRADKVREIAAKLGRHVAILGDLQGPKIRVST
FKEGKVFLNIGDKFLLDANLGKGEGDKEKVGIDYKGLPADVVPGDILLLD
DGRVQLKVLEVQGMKVFTEVTVGGPLSNNKGINKLGGGLSAEALTEKDKA
DIKTAALIGVDYLAVSFPRCGEDLNYARRLARDAGCDAKIVAKVERAEAV
CSQDAMDDIILASDVVMVARGDLGVEIGDPELVGIQKALIRRARQLNRAV
ITATQMMESMITNPMPTRAEVMDVANAVLDGTDAVMLSAETAAGQYPSET
VAAMARVCLGAEKIPSINVSKHRLDVQFDNVEEAIAMSAMYAANHLKGVT
AIITMTESGRTALMTSRISSGLPIFAMSRHERTLNLTALYRGVTPVHFDS
ANDGVAAASEAVNLLRDKGYLMSGDLVIVTQGDVMSTVGSTNTTRILTVE
Lactate dehydrogenase (LDH)(SEQ ID NO 2):
MGSSHHHHHHSSGLVPRGSHMKLAVYSTKQYDKKYLQQVNESFGFELEFF
DFLLTEKTAKTANGCEAVCIFVNDDGSRPVLEELKKHGVKYIALRCAGFN
NVDLDAAKELGLKVVRVPAYDPEAVAEHAIGMMMTLNRRIHRAYQRTRDA
NFSLEGLTGFTMYGKTAGVIGTGKIGVAMLRILKGFGMRLLAFDPYPSAA
ALELGVEYVDLPTLFSESDVISLHCPLTPENYHLLNEAAFDQMKNGVMIV
NTSRGALIDSQAAIEALKNQKIGSLGMDVYENERDLFFEDKSNDVIQDDV
FRRLSACHNVLFTGHQAFLTAEALTSISQTTLQNLSNLEKGETCPNELV

In view of the multimeric structures of PykA and LDH (homotetramers), each enzyme will display multiple pendant (His)₆ tags, which, in turn, will function to crosslink with the QDs and/or NPLs forming nanoclustered or nanoaggregate structures. These are the structures that provide the coupled enzymatic systems with the necessary localized density to engage in channeled catalytic flux. When the QDs or NPLs are mixed with such multimeric enzymes in a solution, they will self-assemble and form nanoclusters following a diffusion-limited aggregation mechanism (DLA). Classical DLA is a process whereby particles diffusing due to Brownian motion follow a random walk path and then cluster together and form aggregates as they interact. In the exemplary scenario, the DLA process is mechanistically the same but there are now two participants, with each displaying one component of the necessary binding interaction—the (His)₆ tag or the receptive ZnS surface. The number of variables involved including NP size/shape, the number of enzymes, the enzyme's size/shape, reaction volume, reaction concentrations, ratios of protein to NP, etc., means that this is not a process that can be accurately modeled or simulated at the current time. Switching to different enzymes or adding more upstream/downstream enzymes all just increase the resulting complexity without even considering use of mixed NP systems. Moreover, the nanoaggregates that form will be an ensemble of different sizes with each having a different number of component NPs and enzymes present. Nevertheless, some control of nanocluster size can be afforded by relative ratio of NP to overall enzyme present with increased NP presence giving rise to larger clusters. Larger clusters also in general manifest a high level of channeled catalytic flux since more enzymes are present in each cluster in close proximity to each other. In general, higher protein concentration over NP means smaller clusters as the proteins will now surround individual NPs with less chance of crosslinking. In contrast, higher NP concentration versus protein means larger clusters as the proteins more readily bind between the NPs and crosslink them. Thus, control over the relative ratio of NP to enzyme present represents a rudimentary control knob over the rate of channeled flux that can be attained in the clusters.

The interaction between enzymes and nanoparticles in these mixed systems demonstrates complex behavior that directly impacts catalytic performance. The metal affinity coordination between histidine tags and zinc-containing surfaces provides the primary attachment mechanism, but secondary interactions also play important roles. The surface curvature of quantum dots and the planar nature of nanoplatelets create different local environments that influence enzyme orientation and stability.

The multimeric nature of both PykA and LDH provides multiple attachment points that enable crosslinking between different nanoparticles. This crosslinking behavior differs between quantum dots and nanoplatelets due to their distinct geometries. The quantum dots tend to create point-to-point connections, while nanoplatelets can accommodate multiple attachment sites across their surface. The combination of these different attachment patterns in mixed systems creates unique structural arrangements that enhance stability and catalytic performance.

The local environment around enzymes attached to different nanoparticle surfaces also influences their activity. Enzymes attached to quantum dots experience a different local ionic strength and pH environment compared to those attached to nanoplatelets. These differences contribute to the enhanced performance of mixed systems by providing multiple microenvironments that can accommodate different optimal conditions for each enzyme in the cascade.

The formation of enzyme-nanoparticle clusters follows a diffusion-limited aggregation (DLA) mechanism that cannot be accurately simulated due to multiple variables including: NP size/shape, number of enzymes present, enzyme size/shape, reaction volume/concentrations, ratios of protein to NP, and other factors. The nanoaggregates form as an ensemble of different sizes, with each having different numbers of component NPs and enzymes present. Control over cluster size can be achieved through the relative ratio of NP to overall enzyme present—higher NP presence gives rise to larger clusters which generally manifest higher levels of channeled catalytic flux since more enzymes are present in close proximity. Higher protein concentration over NP results in smaller clusters as proteins surround individual NPs with less crosslinking opportunity. Higher NP concentration versus protein leads to larger clusters as proteins more readily bind between NPs and crosslink them.

Analytical characterization of the mixed nanoparticle-enzyme clusters reveals complex structural organization that correlates with enhanced performance. Transmission electron microscopy (TEM) shows distinct morphological features depending on the ratio of quantum dots to nanoplatelets. At optimal ratios, the clusters display organized structures where quantum dots appear to bridge between nanoplatelet surfaces, creating three-dimensional networks that maintain colloidal stability while achieving high local enzyme concentrations.

Dynamic light scattering measurements confirm that mixed systems achieve optimal cluster sizes between 50-200 nm, depending on specific composition. These clusters remain stable in solution while providing sufficient local concentration for efficient channeling. The ability to maintain stable clusters in this size range represents a significant advantage over single-particle systems, which often form larger aggregates that can lead to precipitation.

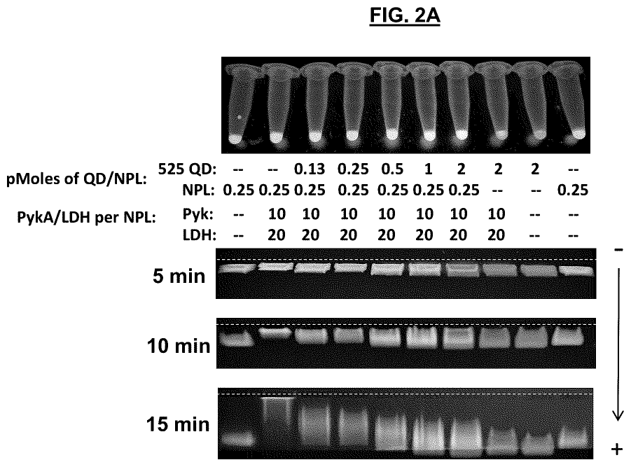

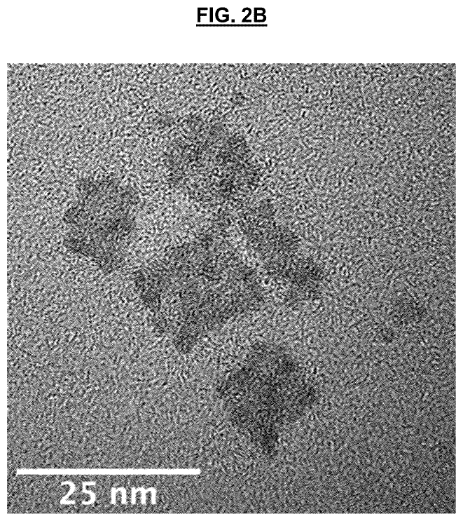

FIGS. 2A-2B illustrate mixed nanoparticle-enzyme clusters formed with the QDs and NPLs. The top portion of FIG. 2A shows 525 nm emitting QDs (green) and 585 nm emitting NPLs (orange) assembled with the ratios of PykA and LDH indicated at increasing ratios of the 525 QDs relative to fixed NPL concentration. Samples are shown in the Eppendorf tubes that they were assembled in with the resulting photoluminescent color indicative of the amount of QD or NPL present. The bottom portion of FIG. 2A shows the samples separated in a 0.85% agarose gel run in 1×TBE buffer. Samples were separated using ca. 10 V per cm gel length and the run stopped every 5 mins to collect an image using a cellphone camera. The location of the wells is indicated by the white dashed line. Fluorescent images collected on a UV-trans-illuminator with 365 nm excitation. FIG. 2B is a representative TEM micrograph from a sample mixture containing nanoclusters formed from LDH (40 nM), PykA (20 nM), 525 QDs (1 nM), and NPLs (0.38 nM) present.

Agarose gel mobility shift assays were used to confirm individual enzyme and joint bienzyme cascade assembly with either the QD series or the NPLs in a manner similar to that described previously. Low electroendosmosis (EEO) agarose gels were utilized with percentages as indicated in each image set. The percentage agarose in each gel was varied as needed to obtain separation. Tris/Borate/EDTA (TBE) buffer (89 mM Tris, 89 mM boric acid, 2 mM EDTA pH 8.3) was used unless otherwise indicated. Images were collected at 5-minute intervals during separation to show the evolution of mobility differences with time. QDs and NPLs were assembled with PykA and LDH both individually and together and then were imaged as they were subsequently separated in an agarose gel under an electrical field. Increasing enzyme assembly to the NPs will decrease the migration rate in a manner that is somewhat proportional to the underlying ratios. The degree of NP mobility shift is distinctly different when each enzyme is present individually, and as a cascaded assembly, confirming cluster formation under both conditions. Again, FIG. 2A shows representative results where different concentrations of 525 QDs (green) and a fixed concentration of 585 NPLs (orange) were assembled with the indicated ratios of PykA and LDH (given as the ratio per NPL). The top image shows the samples as preassembled in Eppendorf tubes where the resulting color is indicative of the amount of QD (green) or NPL (orange) present. The bottom gel images show the samples as separated in a 0.85% agarose gel at different time-periods. Mixed QD-NPL samples separate in the gel with a very different migration rate than either alone and this rate changes as the ratio of 525 QD to 585 NPL changes again confirming assembly of the constructs at different ratios.

TEM was further utilized to image and semi-quantitatively characterize the relative size of the clusters that formed and how these are subsequently altered as the ratio of NP to enzyme is increased. Clusters are defined as NPs that appear to be co-assembled together with a separation distance less than or equal to the size of the NP itself. Clusters are binned or defined by the number of NPs present in each. Results suggest that while the cluster distribution remains similar across the different QD sizes, the use of the NPLs enable larger sized clusters to form at lower concentrations than that used for the QDs. This data is similar to what was previously seen with the NPLs when they were assembled with a 7 enzyme cascade drawn from oxidative glycolysis. Lastly, TEM data is obtained to confirm mixed NP cluster formation from a mixture containing LDH (40 nM) and PykA (20 nM) with both the 525 QDs (1 nM) and NPLs (0.38 nM) present. As shown in FIG. 2B, successful formation of mixed NP clusters was confirmed. These types of mixed NP systems were not quantified by analyzing cluster distributions due to the complexity of the resulting clusters. However, these data do still confirm that clusters containing both 525 QDs and NPLs successfully form in the self-assembly of this two-enzyme cascade.

Characterization of the mixed nanoparticle-enzyme clusters employed multiple complementary techniques. Beyond the TEM imaging described above, dynamic light scattering (DLS) measurements provided information about cluster size distributions in solution. Gel electrophoresis mobility shifts not only confirmed assembly but also provided insights into cluster density and composition through careful analysis of migration patterns.

The agarose gel electrophoresis characterization was performed using 0.85% low electroendosmosis agarose in 1×TBE buffer (89 mM Tris, 89 mM boric acid, 2 mM EDTA pH 8.3). Gels were run at 10V per cm gel length with images collected every 5 minutes. Samples were visualized using both visible light for general protein/nanoparticle migration and UV excitation at 365 nm for tracking quantum dot fluorescence. Migration distances were quantified relative to free nanoparticle controls run in adjacent lanes.

The photoluminescence properties of both QDs and NPLs served as useful probes of cluster formation and stability. Changes in emission intensity and peak position correlate with assembly state and cluster density. Furthermore, the distinct emission wavelengths of different nanoparticle components allowed for simultaneous monitoring of multiple cluster components.

UV-visible spectroscopy enabled real-time monitoring of enzyme activity through NADH absorption measurements while also providing information about cluster formation through scattering measurements. The combination of these various characterization methods provided comprehensive understanding of both structure and function in these complex assemblies.

The photoluminescence properties of both QDs and NPLs served as useful probes of cluster formation and stability. Changes in emission intensity and peak position correlate with assembly state and cluster density. Furthermore, the distinct emission wavelengths of different nanoparticle components allowed for simultaneous monitoring of multiple cluster components.

UV-visible spectroscopy enabled real-time monitoring of enzyme activity through NADH absorption measurements while also providing information about cluster formation through scattering measurements. The combination of these various characterization methods provided comprehensive understanding of both structure and function in these complex assemblies.

TEM imaging utilized plasma-cleaned grids (Ultrathin Carbon Film on Lacey Carbon Support Film, 400 mesh, Copper) prepared by sequential application of: 1) poly-l-lysine solution (10 μL, 0.1 mg/mL) for 1 minute; 2) water rinse; 3) sample application (5 μL) for 10 minutes; and 4) final water rinse to remove excess salt. This methodology enabled clear visualization of both individual nanoparticles and assembled clusters.

The kinetic profiles for PykA and LDH, both free in solution and when NP-displayed (on-QD or on-NPL) at different ratios, were next characterized. Michaelis Menten (MM) assay formats using excess substrate ([S]>>>[E]) to meet standard Briggs-Haldane expectations were applied. While the active QD-enzyme clusters do not meet all the strictest definitions of the MM formalism, values derived from this analysis provide a useful basis for comparison between free enzyme performance and on-NP assays, however, all reported values are qualified as ‘apparent’. Table 1 and Table 2 list the MM descriptors for PykA and LDH, respectively, including the maximal velocity (Vₘₐₓ), catalytic rate (k_{cat}), Michaelis constant (K_{M}), and the k_{cat}/K_{M} ratio, which is a second order rate constant giving the kinetic efficiency—sometimes referred to as the specificity constant. Assays were carried out with equal concentrations of each enzyme free in solution and then as assembled with ratios of 1, 2, 4, and 8 enzymes/NP. The assays monitored changes to NAD⁺ formation via absorbance on a microtiter plate reader either directly from the enzyme in question or in a coupled enzyme format.

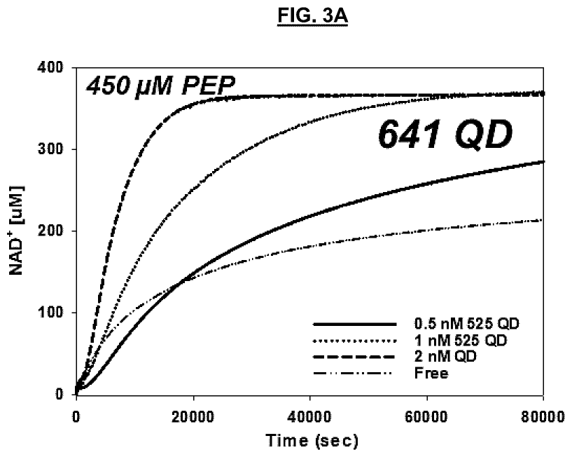

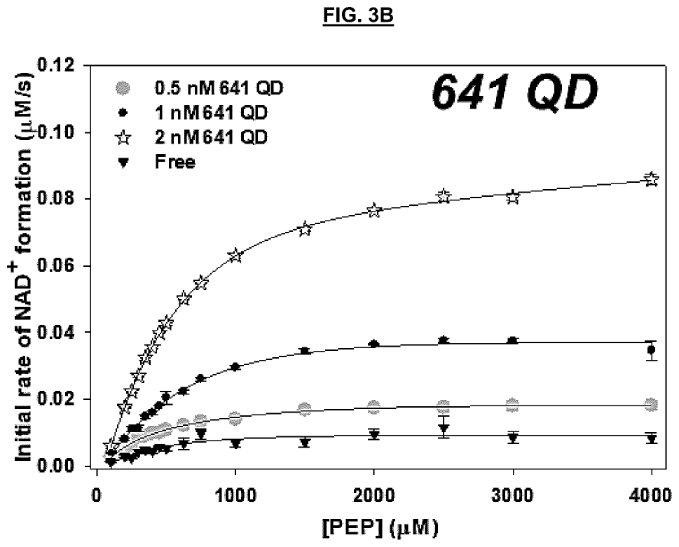

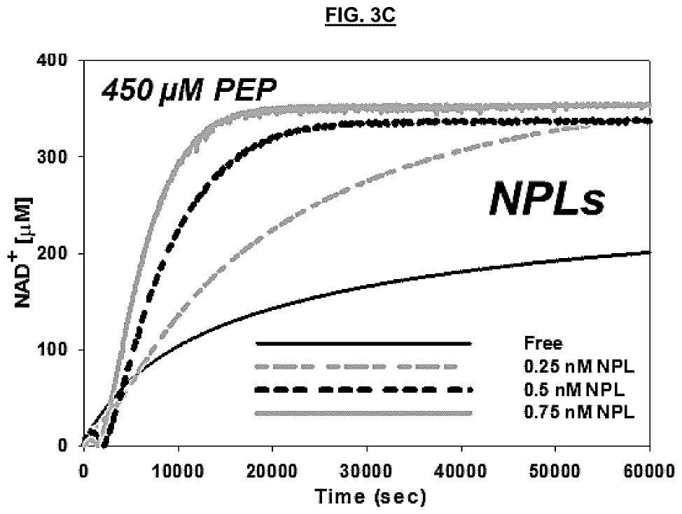

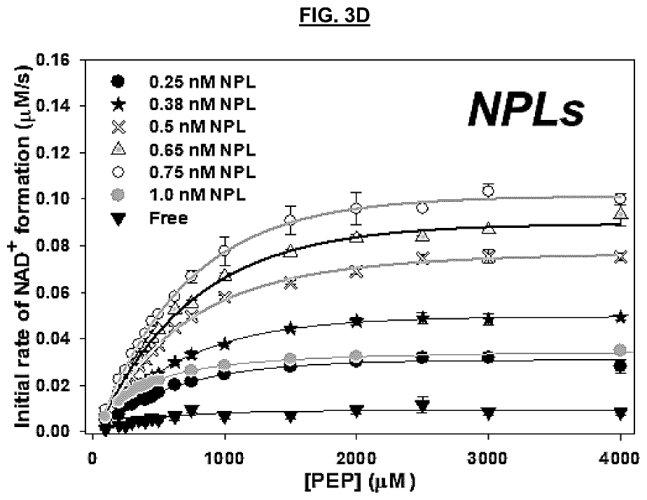

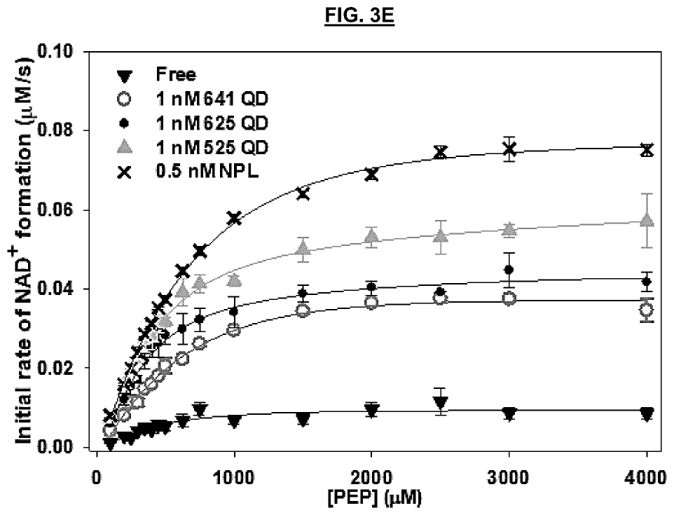

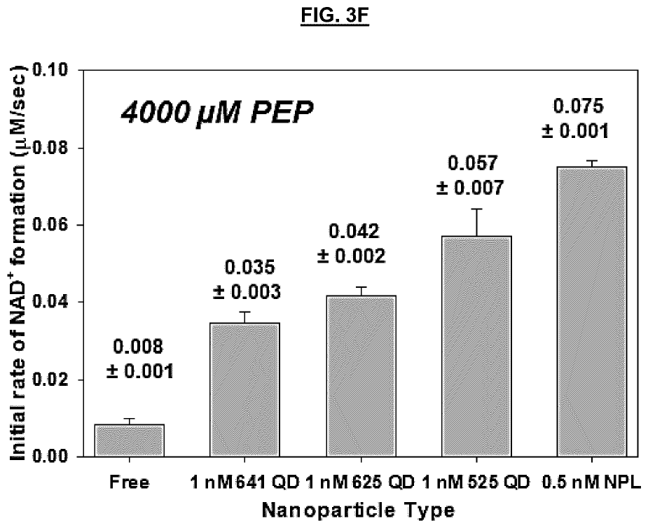

FIGS. 3A-3F illustrate the kinetic enhancement from channeling in the two-enzyme cascade across different individual nanoparticles. FIG. 3A are representative progress curves of NAD⁺ concentration versus time for the two-enzyme cascade at increasing concentrations of 641 QD with 450 μM PEP. FIG. 3B are plots of k_{flux} showing initial rates of NAD⁺ conversion for the two-enzyme cascade across increasing amounts of 641 QD used in the self-assembly versus increasing concentrations of PEP. FIG. 3C are representative progress curves of NAD⁺ concentration versus time for the two-enzyme cascade at increasing concentrations of NPL with 450 μM PEP. FIG. 3D are progress curves of k_{flux} showing initial rates of NAD⁺ conversion for the two-enzyme cascade across increasing amounts of NPL used in the self-assembly versus increasing concentrations of PEP. Enzyme concentration held constant in each assay while QD concentration varied. FIG. 3E are plots of k_{flux} showing initial rates of NAD⁺ conversion for the two-enzyme cascade comparing the free enzymes with the individual 525 QDs, 625 QDs, 641 QDs, and NPLs used in the self-assembly versus increasing concentrations of PEP. FIG. 3F is a plot of the initial rate of NAD⁺ formation at 4000 μM PEP comparing the free enzymes versus the individual 525 QDs, 625 QDs, 641 QDs, and NPLs used in the self-assembly of PykA and LDH. Enzyme concentrations held constant in each assay. Data points from replicate samples and standard deviations were <15% in all cases. Trend lines to aid the eye are included and are not necessarily the MM fits.

The coupled enzyme kinetic assays were conducted at room temperature (22-25° C.) in a 384-well microplate format with 40 μL total reaction volume per well. Each reaction contained 20 μL of enzyme-nanoparticle solution and 10 μL substrate solution. Progress curves were monitored by measuring absorbance at 340 nm to track NADH consumption using a Tecan Spark® plate reader. Data points were collected every 26 seconds after 3 seconds of orbital shaking. Initial rates were calculated from the linear portions of the progress curves, typically the first 10-15% of substrate conversion.

Individual reaction components were pre-equilibrated to assay temperature before mixing. The 384-well plates were sealed with optical film to prevent evaporation during extended measurements. Background absorbance and inner filter effects were corrected using appropriate controls. Standard curves for NADH were generated daily to account for any instrument variation. Data analysis employed initial rate measurements from the first 10% of substrate conversion to ensure steady-state conditions.

TABLE 1
Estimated enzymatic kinetic parameters for PykA when
free in solution and as assembled on QDs and NPLs.

| Enzyme: | V_{Max} | k_{cat} | K_{M} | k_{cat}/K_{M} |
|---|---|---|---|---|
| Ratio per QD | (nM × s⁻¹) | (sec⁻¹) | (mM) | (mM⁻¹ × s⁻¹) |
| PykA:^{a} 0 | 63.0 ± 1 | 25.0 ± 0.1 | 1.25 ± 0.1 | 2.0 × 10⁻⁵ ± 2 × 10⁻⁶ |
| 525 QDs | | | | |
| 1 | 17.0 ± 1 | 6.8 ± 0.1 | 1.30 ± 0.1 | 5.3 × 10⁻⁶ ± 5 × 10⁻⁷ |
| 2 | 14.0 ± 1 | 5.7 ± 0.1 | 1.40 ± 0.1 | 4.1 × 10⁻⁶ ± 4 × 10⁻⁷ |
| 4 | 21.0 ± 1 | 8.5 ± 0.1 | 1.60 ± 0.1 | 5.3 × 10⁻⁶ ± 3 × 10⁻⁸ |
| 8 | 6.8 ± 0.2 | 2.7 ± 0.1 | 1.70 ± 0.2 | 1.6 × 10⁻⁶ ± 2 × 10⁻⁷ |
| 625 QDs | | | | |
| 1 | 6.9 ± 0.2 | 2.7 ± 0.1 | 1.60 ± 0.1 | 1.7 × 10⁻⁶ ± 8 × 10⁻⁸ |
| 2 | 5.4 ± 0.2 | 2.2 ± 0.1 | 2.00 ± 0.2 | 1.1 × 10⁻⁶ ± 1 × 10⁻⁷ |
| 4 | 3.9 ± 0.1 | 1.6 ± 0.1 | 1.80 ± 0.3 | 8.8 × 10⁻⁷ ± 2 × 10⁻⁷ |
| 8 | 5.7 ± 0.2 | 2.3 ± 0.1 | 1.80 ± 0.2 | 1.3 × 10⁻⁶ ± 1 × 10⁻⁷ |
| 641 QDs | | | | |
| 1 | 2.7 ± 0.2 | 1.1 ± 0.1 | 1.40 ± 0.1 | 7.7 × 10⁻⁷ ± 9 × 10⁻⁸ |
| 2 | 2.6 ± 0.1 | 1.1 ± 0.1 | 1.50 ± 0.1 | 7.0 × 10⁻⁷ ± 2 × 10⁻⁸ |
| 4 | 3.0 ± 0.1 | 1.2 ± 0.1 | 1.80 ± 0.2 | 6.6 × 10⁻⁷ ± 9 × 10⁻⁸ |
| 8 | 2.6 ± 0.1 | 1.0 ± 0.1 | 1.50 ± 0.1 | 6.9 × 10⁻⁷ ± 6 × 10⁻⁸ |
| NPLs | | | | |
| 1 | 17.0 ± 1 | 6.8 ± 0.1 | 1.50 ± 0.1 | 4.5 × 10⁻⁶ ± 4 × 10⁻⁷ |
| 2 | 16.0 ± 1 | 6.4 ± 0.1 | 1.50 ± 0.2 | 4.2 × 10⁻⁶ ± 5 × 10⁻⁷ |
| 4 | 12.0 ± 1 | 5.0 ± 0.1 | 1.70 ± 0.2 | 2.9 × 10⁻⁶ ± 4 × 10⁻⁷ |
| 8 | 12.0 ± 1 | 5.0 ± 0.1 | 1.50 ± 0.1 | 3.2 × 10⁻⁶ ± 2 × 10⁻⁷ |

Final enzyme concentration:
^{a}PykA = 2.5 nM.
All kinetic values are qualified as apparent.
Ratio of 0 = free enzyme in solution, no QD present.

TABLE 2
Estimated enzymatic kinetic parameters for LDH when
free in solution and as assembled on QDs and NPLs.

| Enzyme: | V_{Max} | k_{cat} | K_{M} | k_{cat}/K_{M} |
|---|---|---|---|---|
| Ratio per QD | (nM × s⁻¹) | (sec⁻¹) | (mM) | (mM⁻¹ × s⁻¹) |
| LDH:^{a} 0 | 33.0 ± 3 | 13.3 ± 0.2 | 0.9 ± 0.4 | 1.5 × 10⁻⁵ ± 7 × 10⁻⁶ |
| 525 QDs | | | | |
| 1 | 40.0 ± 6 | 15.9 ± 0.4 | 1.10 ± 0.2 | 1.5 × 10⁻⁵ ± 5 × 10⁻⁷ |
| 2 | 45.0 ± 1 | 17.9 ± 0.1 | 0.9 ± 0.4 | 2.0 × 10⁻⁵ ± 7 × 10⁻⁶ |
| 4 | 40.0 ± 1 | 15.5 ± 0.1 | 1.10 ± 0.3 | 1.4 × 10⁻⁵ ± 4 × 10⁻⁶ |
| 8 | 57.0 ± 1 | 22.7 ± 0.1 | 1.50 ± 0.4 | 1.5 × 10⁻⁵ ± 3 × 10⁻⁶ |
| 625 QDs | | | | |
| 1 | 69.0 ± 1 | 27.6 ± 0.2 | 1.30 ± 0.3 | 2.1 × 10⁻⁵ ± 5 × 10⁻⁶ |
| 2 | 58.0 ± 3 | 23.1 ± 0.2 | 1.00 ± 0.2 | 2.3 × 10⁻⁵ ± 4 × 10⁻⁶ |
| 4 | 49.0 ± 1 | 19.5 ± 0.1 | 1.10 ± 0.3 | 1.8 × 10⁻⁵ ± 5 × 10⁻⁶ |
| 8 | 52.0 ± 1 | 20.7 ± 0.1 | 1.20 ± 0.3 | 1.7 × 10⁻⁵ ± 5 × 10⁻⁶ |
| 641 QDs | | | | |
| 1 | 8.3 ± 0.1 | 3.32 ± 0.1 | 0.55 ± 0.2 | 6.0 × 10⁻⁶ ± 2 × 10⁻⁶ |
| 2 | 7.7 ± 0.3 | 3.07 ± 0.1 | 0.80 ± 0.1 | 3.8 × 10⁻⁶ ± 7 × 10⁻⁷ |
| 4 | 3.6 ± 0.5 | 1.45 ± 0.1 | 0.7 ± 0.3 | 2.1 × 10⁻⁶ ± 1 × 10⁻⁶ |
| 8 | 2.6 ± 0.3 | 1.04 ± 0.1 | 0.8 ± 0.1 | 1.3 × 10⁻⁶ ± 3 × 10⁻⁷ |
| NPLS | | | | |
| 1 | 81.0 ± 9 | 32.3 ± 0.6 | 1.60 ± 0.3 | 2.0 × 10⁻⁵ ± 6 × 10⁻⁶ |
| 2 | 71.0 ± 4 | 28.6 ± 0.3 | 0.168 ± 0.2 | 1.7 × 10⁻⁵ ± 2 × 10⁻⁶ |
| 4 | 59.0 ± 5 | 23.6 ± 0.3 | 1.70 ± 0.4 | 1.4 × 10⁻⁵ ± 4 × 10⁻⁶ |
| 8 | 52.0 ± 2 | 20.9 ± 0.1 | 1.70 ± 0.3 | 1.2 × 10⁻⁵ ± 3 × 10⁻⁶ |

Final enzyme concentration:
^{a}LDH = 2.5 nM.
All kinetic values are qualified as apparent.
Ratio of 0 = free enzyme in solution, no QD present.

As can be seen in Table 1, for PykA the catalytic rate (k_{cat}=25 s⁻¹) and efficiency (k_{cat}/K_{M}=2.0×10−5 mM⁻¹s⁻¹) decreased by ˜70% upon NP immobilization. However, immobilization was shown to increase the activity of LDH as compared to the freely diffusing enzyme (data not shown). For free LDH, the catalytic rate (k_{cat}=13.3 s⁻¹) and efficiency (k_{cat}/K_{M}=1.5×10−5 mM⁻¹s⁻¹) were comparable to those of free PykA (Table 2). Upon LDH immobilization onto the 525 QDs, k_{cat} increased to 17.9 s⁻¹ from 13.3 s⁻¹ (˜35%) at the 2 LDH to 1 QD ratio and to 22.7 s⁻¹ (˜72%) at a ratio of 8 LDH. Similarly, upon LDH immobilization onto the 625 QDs, k_{cat} increased to 27.6 s⁻¹ (˜110%) and 23.1 s⁻¹ (76%) at QD display ratios of 1 and 2, respectively. However, upon LDH immobilization onto the 641 QDs, k_{cat} decreased to 3.1 s⁻¹ (˜86%) at the 2 LDH to 1 QD ratio or worse for other ratios. Optimal conditions for LDH activity were observed when immobilized onto the NPLs, where the k_{cat} increased to 32.3 s⁻¹ at the 1 LDH to 1 NPL ratio (2.4× or 240%). In general, the K_{M} values for PykA and LDH decreased as compared to free enzyme meaning that increases in k_{cat}/K_{M} were mostly not seen. These results are in accordance with previous findings albeit with some variability due to altered assay conditions for enzyme concentration, buffer, and pH. To examine the display of enzymes on mixed NP scaffolds, next examined was the individual activity of Pyka and LDH at a ratio of 2 enzyme per NP in representative samples comprising of three different ratios of 525 QD to NPL, see Table 3. For PykA, k_{cat}=13.3 s⁻¹ was again decreased, but interestingly, not as much as when displayed on either the 525 QDs or NPLs at the same ratio. In contrast, LDH activity was found to be better than all configurations excepting the largest display ratio of 8 LDH to 525 QD and then less than that seen with NPL alone except for the largest ratio of 8 again.

The optimization process for mixed nanoparticle-enzyme systems follows a systematic approach that considers multiple interdependent parameters. Initial screening begins with evaluation of individual nanoparticle performance to establish baseline activities. The process then moves to preliminary mixing studies where different nanoparticle ratios are evaluated while maintaining constant total nanoparticle concentration. This approach allows identification of promising composition ranges for more detailed optimization.

Subsequent fine-tuning of the system requires careful adjustment of multiple parameters simultaneously. The total nanoparticle concentration must be balanced against the desired enzyme loading, while maintaining appropriate ratios between different nanoparticle types. The optimal ratios often depend on the specific properties of the enzymes involved, including their size, number of histidine tags, and catalytic rates.

The assembly process itself requires careful control of conditions including temperature, pH, and ionic strength. These parameters affect not only the initial assembly of the clusters but also their long-term stability and catalytic performance. Monitoring of assembly progress through multiple complementary techniques ensures formation of optimal structures.

The kinetic profiles were analyzed in detail using Michaelis-Menten formalism, though it should be noted that the active QD-enzyme clusters do not meet all strict MM definitions. The assays monitored NAD⁺ formation via absorbance measurements under the following standardized conditions:

Buffer composition: 120 mM HEPES (pH 8) with 1 mM EDTA and 10 mM KCl Stock solutions prepared in buffer:
500 nM PykA stock
5000 nM LDH stock
500 nM nanoparticle stocks
100 mM PEP stock (pH monitored and adjusted to 8)
100 mM ADP stock
800 mM MgCl₂·6H₂O stock
50 mM NADH stock

### Final Assay Concentrations:

2.5 nM PykA or LDH
Variable nanoparticle concentration (0-5 nM)
Variable PEP (0.1-10 mM)
1 mM ADP
10 mM MgCl₂
0.25 mM NADH
0.5 mM EDTA
10 mM KCl

Control experiments were conducted to validate the specificity of the observed effects. These included: Assembly with non-specific proteins to confirm His-tag specificity; Competition experiments with free histidine to demonstrate reversibility; Assembly with surface-blocked nanoparticles to confirm specific binding; Thermal stability studies comparing free and nanoparticle-bound enzymes; Size exclusion chromatography to confirm cluster formation; and Activity measurements in the presence of varying concentrations of chelating agents.

The competition experiments with free histidine were conducted over 24 hours to ensure equilibrium was reached. Activity measurements with chelating agents were performed after pre-incubation periods ranging from 0 to 4 hours to assess both immediate and longer-term effects on cluster stability.

TABLE 3
Estimated enzymatic kinetic parameters for LDH
and PykA for mixed NPL and 525 QD systems.

| Enzyme: | V_{Max} | k_{cat} | K_{M} | k_{cat}/K_{M} |
|---|---|---|---|---|
| Ratio per NP | (nM × s⁻¹) | (sec⁻¹) | (mM) | (mM⁻¹ × s⁻¹) |
| PykA: 2 (2.5 nM) | | | | |
| PykA only | 63.0 ± 1 | 25.0 ± 0.1 | 1.25 ± 0.1 | 2.0 × 10⁻⁵ ± 2 × 10⁻⁶ |
| 1 nM 525 QD/0.25 nM NPL | 24.0 ± 1 | 9.7 ± 0.1 | 1.00 ± 0.1 | 9.8 × 10⁻⁶ ± 8 × 10⁻⁷ |
| 0.75 nM 525 QD/0.5 nM | 25.0 ± 1 | 10.0 ± 0.1 | 1.20 ± 0.2 | 8.3 × 10⁻⁶ ± 1 × 10⁻⁶ |
| NPL | | | | |
| 0.25 nM 525 QD/1 nM NPL | 25.0 ± 2 | 10.0 ± 0.1 | 1.10 ± 0.2 | 9.1 × 10⁻⁶ ± 9 × 10⁻⁷ |
| LDH: 2 (2.5 nM) | | | | |
| LDH only | 33.0 ± 3 | 13.3 ± 0.2 | 0.9 ± 0.4 | 1.5 × 10⁻⁵ ± 7 × 10⁻⁶ |
| 1 nM 525 QD/0.25 nM NPL | 57.0 ± 1 | 23.0 ± 0.1 | 1.30 ± 0.1 | 1.8 × 10⁻⁵ ± 1 × 10⁻⁶ |
| 0.75 nM 525 QD/0.5 nM | 53.0 ± 1 | 21.1 ± 0.1 | 1.10 ± 0.2 | 1.9 × 10⁻⁵ ± 2 × 10⁻⁶ |
| NPL | | | | |
| 0.25 nM 525 QD/1 nM NPL | 53.0 ± 2 | 21.1 ± 0.1 | 1.20 ± 0.2 | 1.8 × 10⁻⁵ ± 4 × 10⁻⁶ |

Final enzyme concentration:
All kinetic values are qualified as apparent.
Ratio of 2 = 2.5 nM enzyme, total mixed QD/NPL concentration 1.25 nM.

In addition to the establishment of kinetic profiles for both PykA and LDH across the four different NPs, the variation of intermediary channeling between these two-enzymes across nanoclusters formed with the individual materials was examined. This involved testing the apparent k_{flux} for the two-enzyme cascade across the 525, 625, and 641 QDs. The NPLs were also examined, but at a lower overall concentration relative to the QDs due to the extended flat surface area of the NPL material and also its propensity towards precipitation at higher working concentrations in clusters Again, these assays monitored NAD⁺ formation via absorbance on a plate reader. Since the catalytic rate of PykA was twice as fast as LDH, all experiments aimed to analyze channeling were done with twice the amount of LDH relative to PykA such that the initial rates of each enzyme were well matched.

The systematic optimization of reaction kinetics in mixed nanoparticle systems revealed several key principles for maximizing cascade efficiency. First, the relative concentrations of PykA and LDH must be carefully balanced to match their catalytic rates. The optimal ratio of 1:2 PykA:LDH ensures that the faster-acting PykA does not overwhelm the capacity of LDH to process the intermediate products. This ratio holds true across different nanoparticle compositions but becomes particularly important in mixed systems where local enzyme concentrations can be higher. Second, the relationship between substrate concentration and enzyme activity follows more complex patterns in mixed nanoparticle systems compared to single-particle or free enzyme systems. The enhanced local concentration of enzymes in optimized clusters allows for efficient substrate processing even at lower bulk substrate concentrations. However, the system must be carefully balanced to avoid substrate depletion effects that could limit overall throughput. Third, the stability of reaction intermediates becomes less critical in well-optimized mixed systems due to more efficient channeling. The reduced diffusion distances and optimized enzyme orientations in mixed QD-NPL clusters allow for rapid transfer of intermediates between active sites, minimizing losses to bulk solution and potential side reactions.

The panels in FIGS. 3A-D show representative data for the 641 QDs and the NPLs at constant enzyme concentration, which corresponded to the worst and best performing materials, respectively, in this case. FIG. 3A highlights traces of NAD⁺ concentration change versus time for the two-enzyme cascade at increasing concentrations of 641 QD with 450 μM PEP while FIG. 3C shows the same data for the NPLs collected with lower NPL concentrations. FIG. 3B plots k_{flux} showing initial rates of NAD⁺ conversion for the two-enzyme cascade across increasing amounts of 641 QD used versus increasing concentrations of PEP while FIG. 3D shows the same data for the NPLs. Analogous data was collected from the 525 and 625 QDs (data not shown). Analyzing the changes in k_{flux} for the two-enzyme cascade assembled to the three different QDs at increasing concentrations while the concentrations of PykA and LDH remained constant across increasing concentrations of PEP (FIG. 3B), it is clear that the overall k_{flux} increases somewhat linearly with increasing QD concentration. These results align well with what we have previously observed, where increasing the concentration of QD induced larger cluster formation and allowed for higher enzyme incorporation per cluster yielding an overall more efficient channeling mechanism.

Using the NPLs in the two-enzyme cascade assembly, NAD⁺ formation overtime showed a dependence on NPL concentration while the overall k_{flux} increased linearly with increasing NPL concentration ranging from 0.25 up to 0.75 nM (FIG. 3D). Notably, at 1 nM NPL there is a clear suppression in activity and overall k_{flux}. It is believed this was the result of forming clusters, which were too large to remain stable in solution hence our trend of adjusting for this by lowering the working NPL concentrations in a manner similar to a previous report (Breger et al., Nature Communications 14 (1), 1757, 2023). Overall, when comparing the four different NP materials, each individual nanomaterial outperforms the activity that can be observed with the free enzymes under otherwise analogous conditions and a distinct relationship between decreasing QD size and increased k_{flux} can be observed when comparing the three QDs at a 1 nM concentration (FIG. 3E). Comparably, half the amount of NPL can be used to achieve a greater overall k_{flux} relative to the three different QDs (FIG. 3E, purple). Analyzing the initial rate data in FIG. 3E at 4,000 μM PEP and plotting the observed initial rate for the free enzyme system and NP-assembled systems, a linear correlation exits from the free enzyme system through the three QDs of decreasing size (641→625→525 QD) at 1 nM concentration to the NPLs at a concentration of 0.5 nM, where the largest initial rate is observed (FIG. 3F). For the NPLs, the initial rate is enhanced >8× that of the free enzyme.

The two-enzyme cascade was then combined with both QDs and NPLs into mixed-NP assemblies to ascertain if it were possible to achieve a greater enhancement in channeling relative to that obtained with any of the single nanomaterial assemblies. The overarching goal was to identify if these assemblies would even optimize the underlying geometric packing and assembly components to create immobilized enzyme clusters that are even further enhanced for product formation beyond the current strategy of using singular nanomaterial types as the scaffolding that provides enzyme crosslinking. To do this, a series of multi-parametric experiments testing the different NP-enzyme assembly combinations was performed.

First, the individual different sized QDs were combined with the NPLs for the self-assembly of the PykA-LDH system. Having already observed that lower amounts of the NPL could be used in this two-enzyme cascade to achieve an enhancement in activity (FIG. 3D), a starting concentration of 0.38 nM NPL was used while systematically altering the self-assembly reactants to incorporate increasing amounts of 525 QD (0-2 nM).

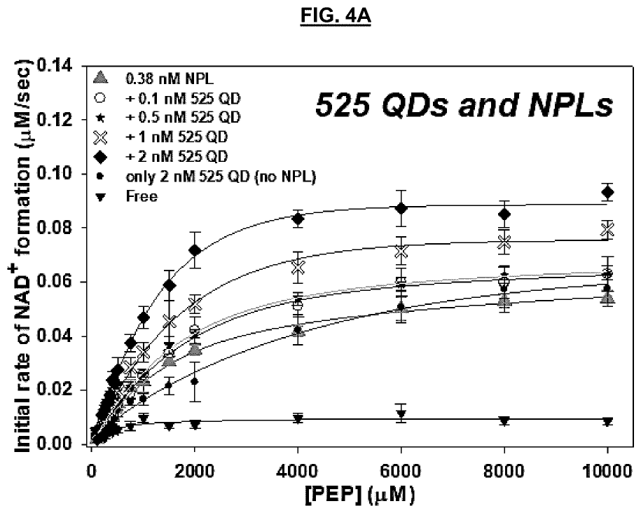

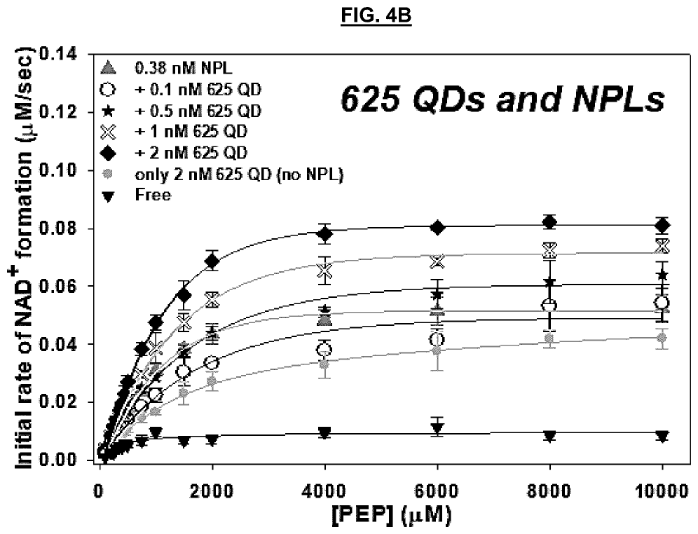

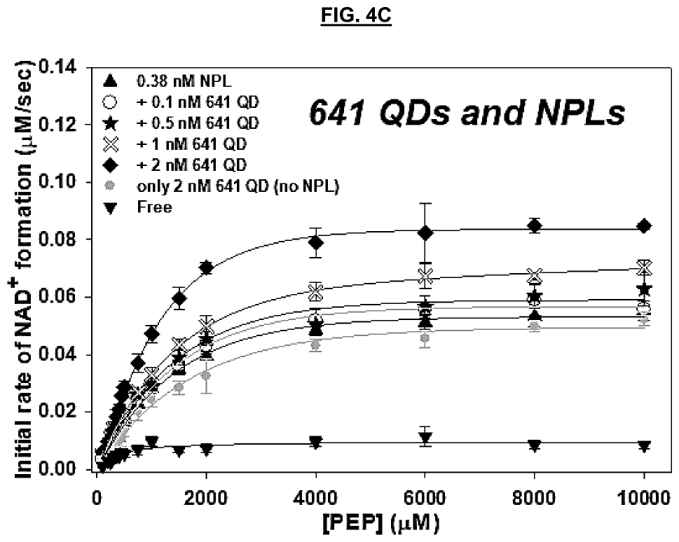

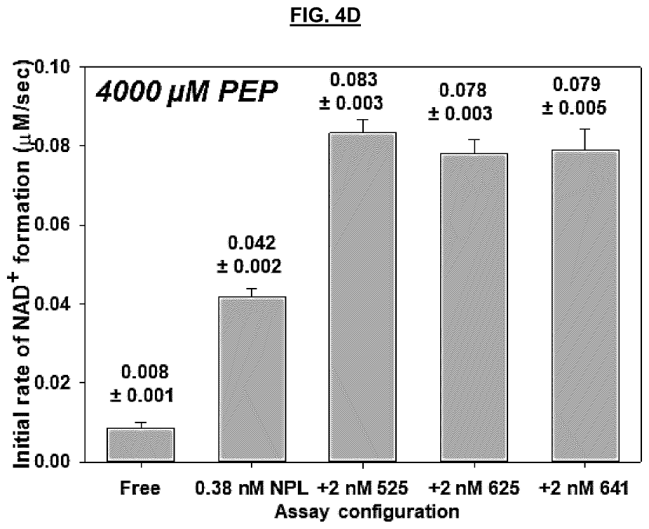

FIGS. 4A-4D show the changes in k_{flux} in the two-enzyme cascade as the result of mixed QD-NPL clusters engaged in channeling. FIG. 4A are plots of k_{flux} showing initial rates of NAD⁺ conversion for the two-enzyme cascade with 0.375 nM NPL and increasing amounts of 525 QD used in the self-assembly versus increasing concentrations of PEP. FIG. 4B are plots of k_{flux} showing initial rates of NAD⁺ conversion for the two-enzyme cascade with 0.375 nM NPL and increasing amounts of 625 QD used in the self-assembly versus increasing concentrations of PEP. FIG. 4C are plots of k_{flux} showing initial rates of NAD⁺ conversion for the two-enzyme cascade with 0.375 nM NPL and increasing amounts of 641 QD used in the self-assembly versus increasing concentrations of PEP. FIG. 4D is a bar graph illustrating the initial rate of NAD⁺ formation that is achieved at 4,000 μM PEP with only 0.38 nM NPL versus 0.38 nM NPL with 2 nM of each of the QDs used in the self-assembly process along with the same concentration of free enzyme. Enzyme concentrations held constant in each assay while NP concentrations varied. Data points from replicate samples and standard deviations were <15% in all cases. Trend lines to aid the eye are included and are not necessarily the MM fits.

As can be observed in FIG. 4A, when the 525 QDs are added increasing the NP concentration beyond the 0.38 nM NPL, a subsequent increase in the overall k_{flux} is observed. This increase in k_{flux} is greater than what is observed with either of the two NPs independently, suggesting that combining the two materials alters cluster formation in such a way that it improves the intermediary channeling process in the two-enzyme cascade. The same data was obtained with the same amount of enzyme and 0.38 nM NPL when adding increasing concentrations of either 625 QD (FIG. 4B) or 641 QD (FIG. 4C) during the NP-enzyme self-assembly process. These results demonstrated a similar trend to what was observed in the 525 QD titration with 0.38 nM NPL. Notably, if one compares the initial rate of the two-enzyme system assembled to 0.38 nM NPL versus that of the two-enzyme system assembled to 0.38 nM NPL mixed with 2 nM of either 525, 625, or 641 QDs at 4,000 μM PEP, one observes a 2× increase in initial rate with the addition of 2 nM QD in the self-assembly process (FIG. 4D). However, this 2× increase in initial rate appears to be independent of QD size, which contrasts with the observation of the independent QD assemblies, described above.

The long-term stability of mixed nanoparticle-enzyme assemblies represents a critical advantage of this approach. These systems maintain catalytic activity under storage conditions for extended periods, with minimal loss of performance over several weeks when stored at 4° C. The enhanced stability stems from several factors inherent to the mixed nanoparticle approach.

The presence of both quantum dots and nanoplatelets provides redundancy in enzyme attachment points, helping maintain cluster integrity even if individual attachment points become compromised. The different geometric arrangements possible in mixed systems also help prevent the formation of overly large aggregates that could lead to precipitation. Furthermore, the mixed nanoparticle environment appears to help stabilize enzyme quaternary structure, particularly for multimeric enzymes like LDH.

Temperature stability studies demonstrate that mixed nanoparticle systems maintain activity over a broader temperature range compared to either single-particle systems or free enzymes. Similar enhanced stability is observed with respect to pH variations and ionic strength changes. This robust stability makes these systems particularly valuable for industrial applications where maintaining consistent performance under varying conditions is crucial.

The optimization of mixed QD-NPL assemblies revealed several critical parameters for achieving enhanced enzymatic activity. First, the relative concentrations of the different nanoparticle components proved crucial for system stability and performance. The NPL concentration must be maintained at a lower level than the QDs due to the NPLs' larger surface area, with 0.38 nM NPL concentration determined to be optimal. In contrast, QD concentration could be varied over a broader range from 0-2 nM, though the total combined nanoparticle concentration directly impacts the stability of the resulting clusters.

In an example, the optimal performance of the mixed nanoparticle systems includes several parameter ranges. The total nanoparticle concentration is maintained between 0.1-5 nM for two-enzyme systems, while larger enzyme cascades can accommodate concentrations up to 100 nM. The temperature during both assembly and operation may be controlled between 4-37° C., with assembly preferentially performed at lower temperatures and operation at room temperature. The pH can be maintained between 7.5-8.5, with optimal performance typically observed at pH 8.0. Ionic strength may be kept between 50-200 mM to maintain colloidal stability while allowing proper enzyme function. The ratio of enzyme to nanoparticle can be maintained between 1:1 and 8:1, with the optimal ratio depending on the specific enzyme system and desired outcome.

The optimization process followed a systematic approach using a design of experiments methodology. Variables were initially screened using a fractional factorial design to identify the most significant parameters. These were then optimized using response surface methodology with central composite design. The optimization process began by evaluating total nanoparticle concentrations from 0.1-5 nM. This was followed by systematic variation of QD:NPL ratios between 1:5 and 2:1. Enzyme:nanoparticle ratios were tested from 1:1 to 8:1. The buffer conditions were optimized by varying pH from 7.0-9.0 and ionic strength from 50-200 mM. Temperature effects were characterized from 4-37° C., while assembly times ranged from 0.5-24 hours.

Buffer composition played a critical role in maintaining optimal assembly and activity. The pH range of 7.5-8.5 was found to be optimal, with pH 8.0 providing the best balance between assembly stability and enzyme activity. Ionic strength was controlled through careful balance of buffer components, with 120 mM HEPES providing suitable buffering while maintaining colloidal stability. The presence of divalent cations, particularly Mg2+, was essential for enzyme activity but required careful optimization to prevent aggregation at higher concentrations.

Buffer components were prepared as concentrated stock solutions and filtered through 0.22 m membranes before use. The HEPES buffer system was selected after evaluation of multiple buffering agents including Tris, phosphate, and MOPS. Divalent cation concentrations were carefully optimized through systematic variation of Mg2+ levels while monitoring both enzyme activity and cluster stability. Final buffer compositions were adjusted to maintain consistent ionic strength across all experimental conditions.

The size and shape characteristics of the nanoparticles significantly influence assembly performance. Smaller QDs, particularly those emitting at 525 nm, consistently demonstrated superior performance compared to their larger counterparts. The distinctive planar shape of the NPLs provides complementary assembly properties that, when combined with spherical QDs, creates unique geometric packing arrangements that enhance enzyme proximity and activity.

The ratio between QDs and NPLs proved to be another critical factor in optimizing system performance. While ratios ranging from 1:5 to 2:1 QD:NPL showed enhanced activity compared to single-particle systems, the optimal ratio depends on the specific enzyme system being employed. Systems with higher total nanoparticle concentrations require particularly careful adjustment of this ratio to maintain stability and performance.

The conditions under which the assemblies are formed also significantly impact their effectiveness. Sequential addition of components, rather than simultaneous mixing, improves the quality and consistency of cluster formation. Temperature control during the assembly process affects the final structural organization, and an assembly period of 3 hours was found to provide optimal cluster formation across different system compositions.

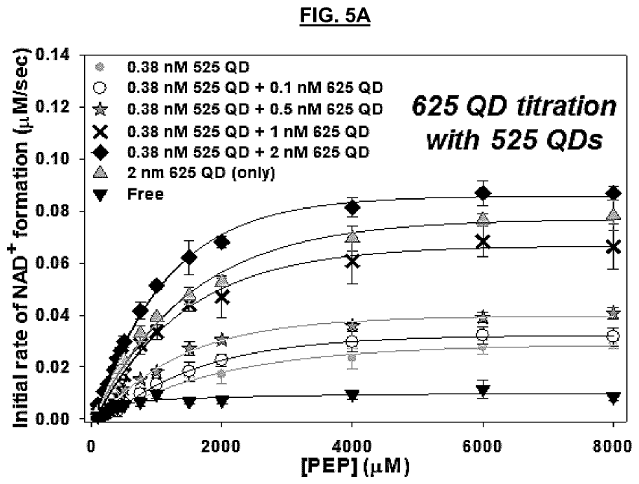

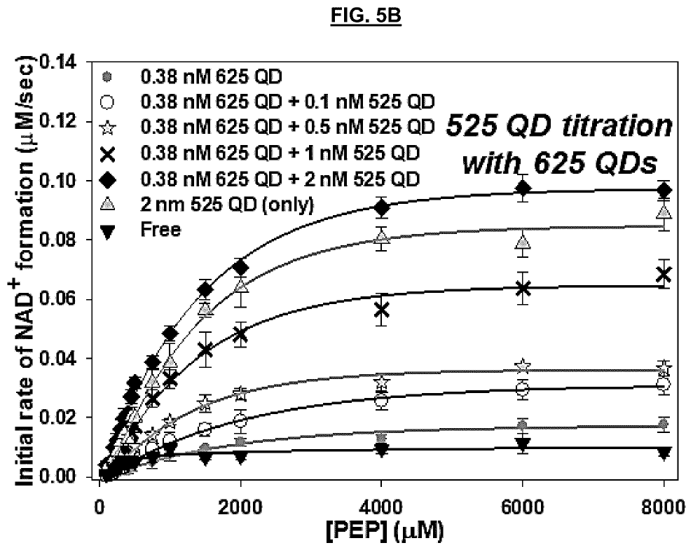

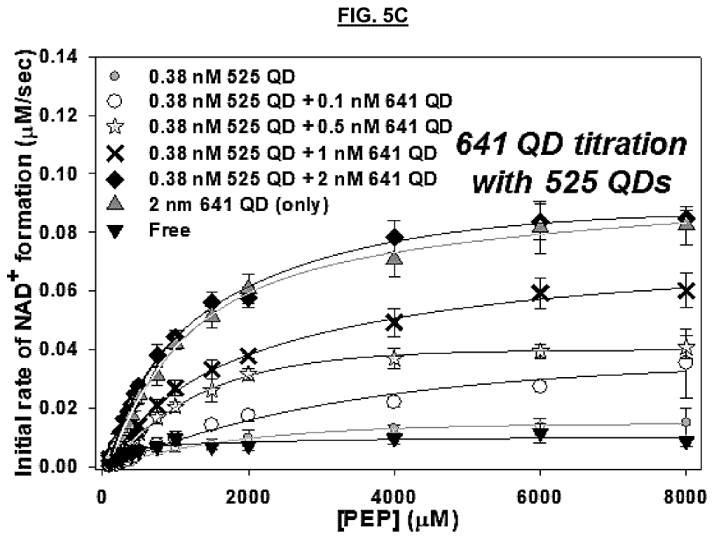

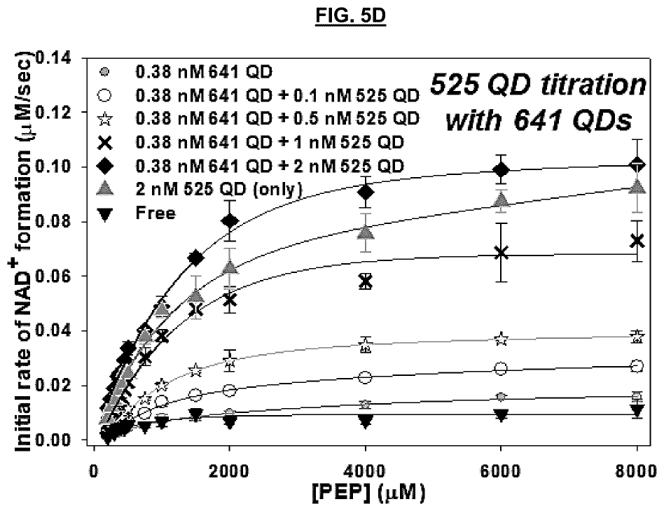

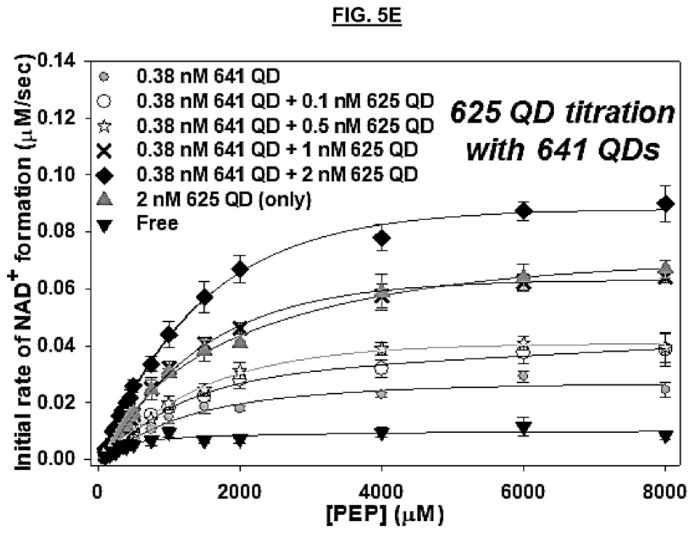

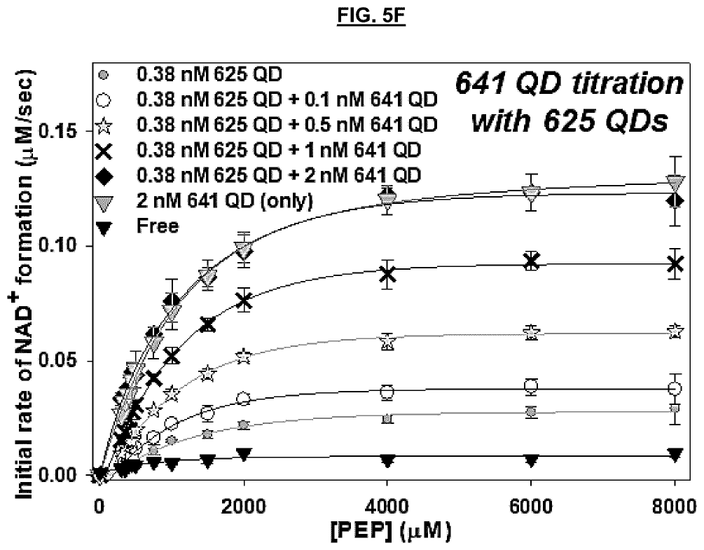

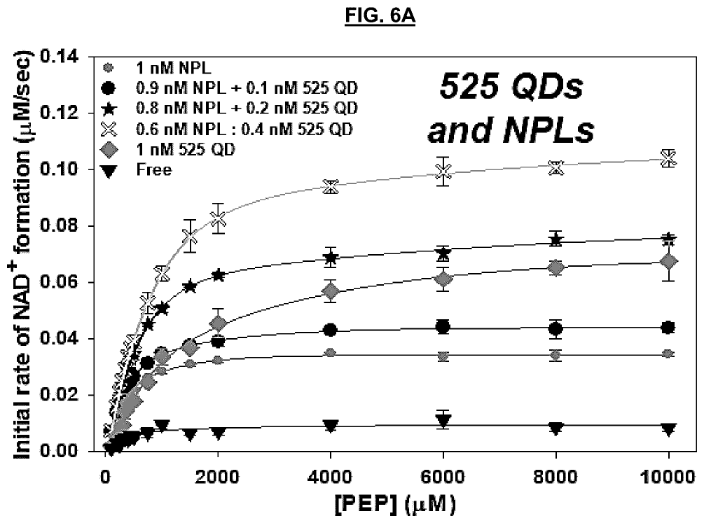

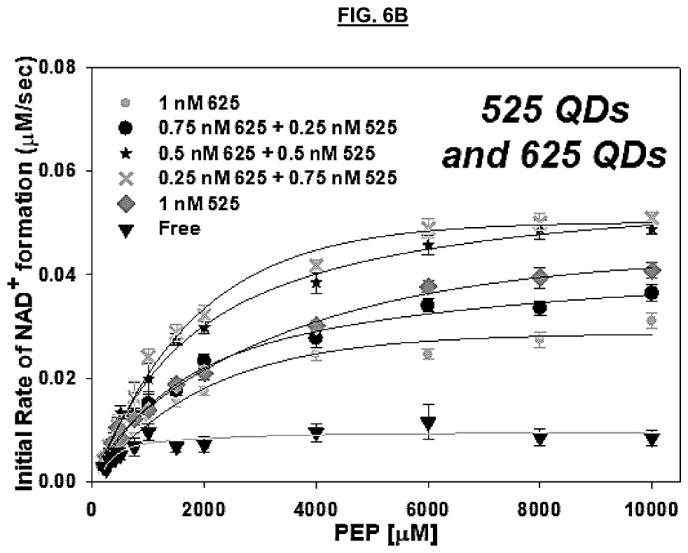

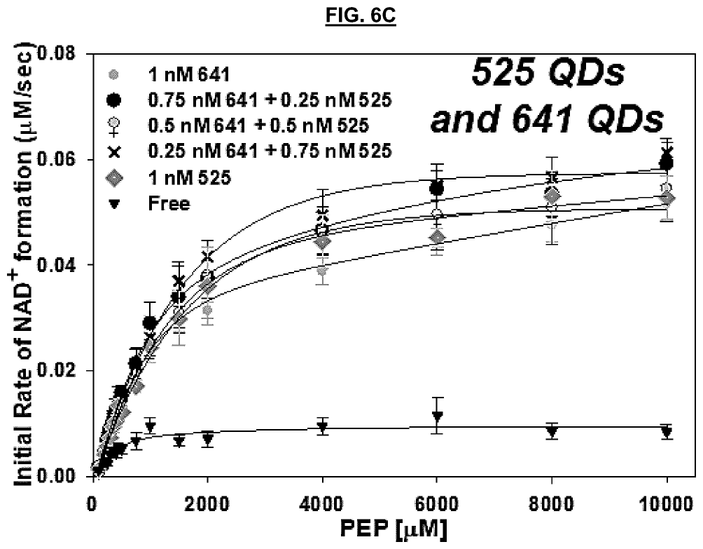

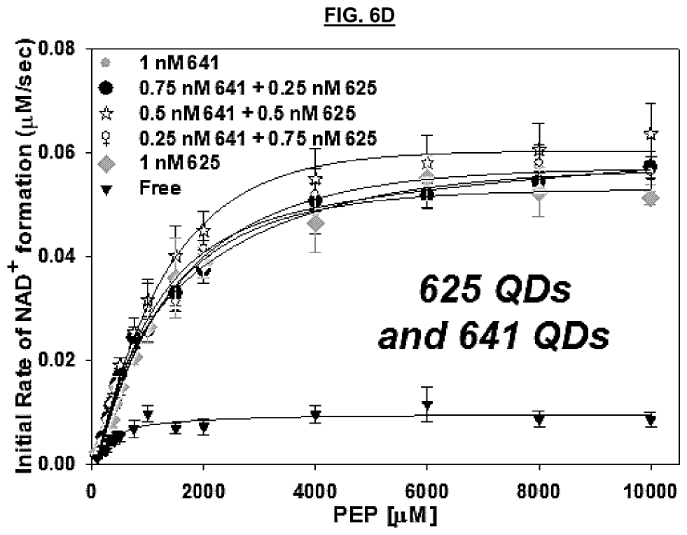

Next, a series of assays were performed attempting to mix the different QD sizes in the self-assembly process in a similar manner as done for the individual QDs and NPLs described above. This was undertaken to see if any combination of differentially-sized QDs in clusters would offer an enhancement in k_{flux} that was greater than when only one QD size was used. These results are illustrated as a series of plots of the initial rate of NAD⁺ formation versus increasing concentrations of PEP shown in FIGS. 5A-F. For each of the three differently sized QDs, a lower concentration of one QD was used (0.38 nM) in the self-assembly process similar to the above for the NPLs with added increasing concentrations of a differently sized QD (0-2 nM). Cumulatively, these results are more suggestive of an effective increase in total nanomaterial concentration causing an enhancement in overall k_{flux}. Intuitively, these results are not unexpected, as under these conditions only the QD sizes are being changed whereas, with the NPLs mixed with differently sized QDs there exists more significant changes in both size and shape, which one would expect should have a greater impact on the geometric packing within the immobilized enzyme cluster formed during the self-assembly process. To further confirm and expand on this, a series of assays were performed where the total concentration of nanomaterial present in the reaction was held constant at 1 nM and then their ratio relative to each was systematically changed (FIGS. 6A-6D). As shown in FIG. 6A, when combining the 525 QDs with the NPLs and maintaining a constant total NP concentration of 1 nM, a distinct enhancement in the overall k_{flux} is created, which is independent of the total amount of NP used in the self-assembly process. However, when the same experiment is performed combining only the QDs of different sizes (FIGS. 6B-D), the overall enhancement in k_{flux} becomes much less pronounced relative to what was observed with the initial NPL-525 QD mix. Overall, the best improvements are obtained when adding NPLs with the QDs in the mixed NP clusters. As was noted previously, the best increases in channeled k_{flux} was obtained with use of the smallest 525 QDs or the NPLs and the same remains true here with individual enzymes, coupled enzymes, and also amongst the mixed QD-NPL systems tested (FIG. 6A). These improvements can be accessed either by increasing the total amount of NP concentration to presumably induce formation of larger nanoclusters or when keeping NP concentration constant but varying the QD-to-NPL ratio present. That such improvements increase beyond what was obtained with NPLs alone also suggests that the mixed NP-enzyme systems are able to extend or increase colloidal stability of the resulting aggregate versus that of NPL alone.

FIGS. 5A-5F show the changes in k_{flux} in the two-enzyme cascade as the result of mixed QDs of different sizes. FIG. 5A show plots of k_{flux} showing initial rates of NAD⁺ conversion for the two-enzyme cascade with 0.38 nM 525 QD and increasing amounts of 625 QD used in the self-assembly versus increasing concentrations of PEP. FIG. 5B show plots of k_{flux} showing initial rates of NAD⁺ conversion for the two-enzyme cascade with 0.38 nM 625 QD and increasing amounts of 525 QD used in the self-assembly versus increasing concentrations of PEP. FIG. 5C show plots of k_{flux} showing initial rates of NAD⁺ conversion for the two-enzyme cascade with 0.38 nM 525 QD and increasing amounts of 641 QD used in the self-assembly versus increasing concentrations of PEP. FIG. 5D show plots of k_{flux} showing initial rates of NAD⁺ conversion for the two-enzyme cascade with 0.38 nM 641 QD and increasing amounts of 525 QD used in the self-assembly versus increasing concentrations of PEP. FIG. 5E show plots of k_{flux} showing initial rates of NAD⁺ conversion for the two-enzyme cascade with 0.38 nM 641 QD and increasing amounts of 625 QD used in the self-assembly versus increasing concentrations of PEP. FIG. 5F show plots of k_{flux} showing initial rates of NAD⁺ conversion for the two-enzyme cascade with 0.38 nM 625 QD and increasing amounts of 641 QD used in the self-assembly versus increasing concentrations of PEP. Enzyme concentrations held constant in each assay while nanoparticle concentrations varied. Full descriptions of each assay are in the SI. Data points from replicate samples and standard deviations were <15% in all cases. Trend lines to aid the eye are included in FIGS. 5A-5F, these are not necessarily the MM fits.

FIGS. 6A-6D show the changes in k_{flux} in the two-enzyme cascade from mixing QDs with NPLs or other QDs at a constant overall concentration. FIG. 6A show plots of k_{flux} showing initial rates of NAD⁺ conversion for the two-enzyme cascade with a constant 1 nM nanoparticle that is varied by mixing 525 QDs and NPLs at different ratios used in the self-assembly versus increasing concentrations of PEP. FIG. 6B show plots of k_{flux} showing initial rates of NAD⁺ conversion for the two-enzyme cascade with a constant 1 nM nanoparticle that is varied by mixing 525 QDs and 625 QDs at different ratios used in the self-assembly versus increasing concentrations of PEP. FIG. 6C show plots of k_{flux} showing initial rates of NAD⁺ conversion for the two-enzyme cascade with a constant 1 nM nanoparticle that is varied by mixing 525 QDs and 641 QDs at different ratios used in the self-assembly versus increasing concentrations of PEP. FIG. 6D show plots of k_{flux} showing initial rates of NAD⁺ conversion for the two-enzyme cascade with a constant 1 nM nanoparticle that is varied by mixing 625 QDs and 641 QDs at different ratios used in the self-assembly versus increasing concentrations of PEP. Enzyme concentrations and overall nanomaterial concentration held constant in each assay while relative nanoparticle ratios to each other varied. Full descriptions of each assay are in the SI. Data points from replicate samples and standard deviations were <15% in all cases. Trend lines to aid the eye are included in FIGS. 6A-6D, these are not necessarily the MM fits.

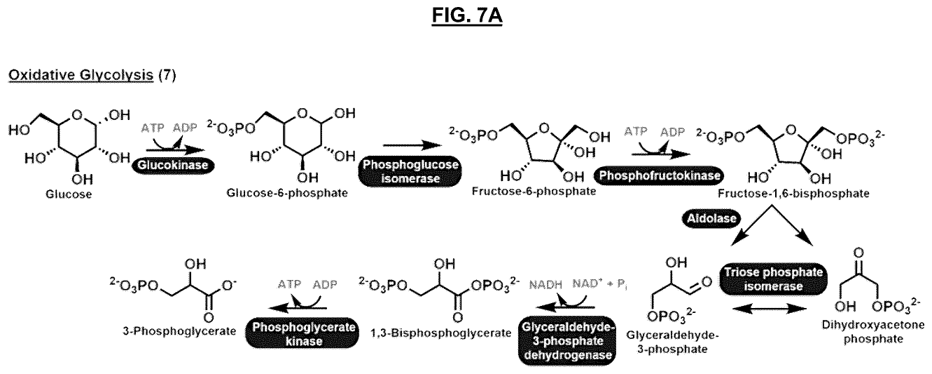

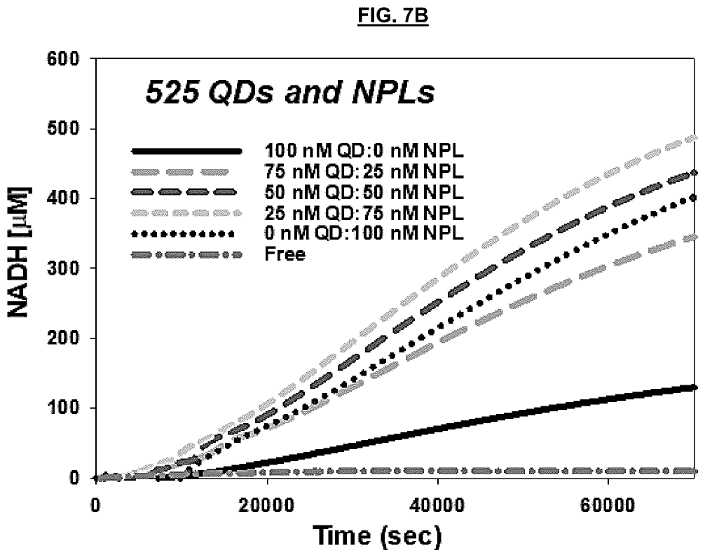

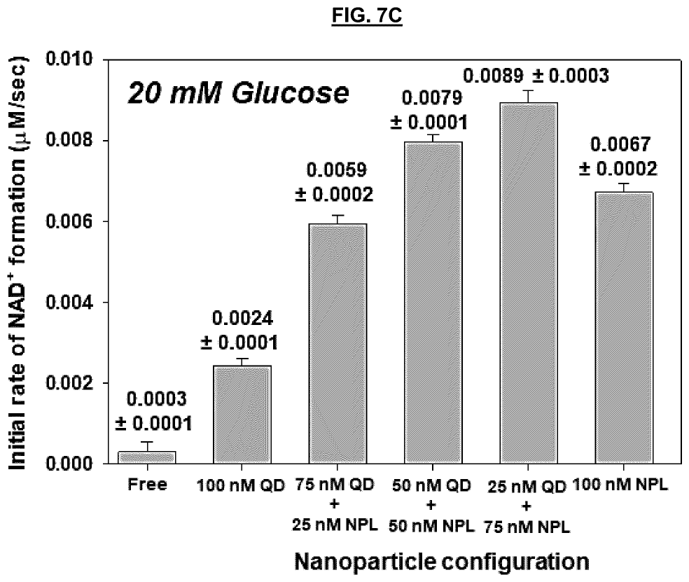

To highlight that the above mixed NP-scaffolded approach has applicability beyond the prototypical two-enzyme system characterized above, initial experiments were conducted with the seven-enzyme cascade that catalyzes the conversion of glucose to 3-phosphoglycerate (3-PG) as part of oxidative glycolysis, as seen in FIG. 7A. This cascade had recently been utilized to extensively characterize the channeling phenomena itself when formed into the requisite NP-enzyme clusters (Breger et al., Nature Communications 14 (1), 1757, 2023). In that report, each experiment used a single NP type for each assay and also confirmed that NPLs could induce more efficient channeling than clusters assembled with spherical QDs. As above, the assay was monitored by measuring the conversion of NAD⁺ to NADH by glyceraldehyde-3-phosphate dehydrogenase (GPD) via changes to the cofactor's absorption at the penultimate enzymatic step on a microtiter well plate reader. The additional conversion step of adding phosphoglycerate kinase into this cascade is due to the strongly negative free energy (ΔG) of this reaction, which helps pull the reaction flux forward vice that of the GPD, which is far more positive and favors the reverse gluconeogenic reaction direction.

FIGS. 7A-7C show the changes in kflux in a seven-enzyme cascade from mixing QDs with NPLs at a constant overall nanoparticle concentration. FIG. 7A shows a seven-enzyme pathway used to convert glucose to 3-phosphoglycrate, → indicates enzymatically catalyzed step(s). Chemical structures of the substrate, intermediaries, and final product shown. FIG. 7B shows progress curves of NADH production for the seven-enzyme cascade with a constant NP concentration of 100 nM that is varied by mixing 525 QDs and NPLs at different ratios used in the self-assembly. Each line represents the average of four replicates. Each line represents the average of four replicates. FIG. 7C shows a plot of initial rate of NAD⁺ production for the seven-enzyme cascade with a constant NP concentration of 100 nM that is varied by mixing 525 QDs and NPLs at different ratios used in the self-assembly. Reaction conditions include 30 mM MgCl2, 15 mM ATP, 15 mM ADP, 20 mM glucose, 8 mM dibasic/monobasic phosphate, 2.25 mM NAD+, and 250 mM HEPES (pH 8). The final concentration of enzymes are as follows: 2.75 nM glucokinase (GlK), 0.5 nM phosphoglucose isomerase (PGI), 4.5 nM phosphofructokinase (PFK), 6 nM fructose-bisphosphate aldolase (FBA), 0.5 nM triose phosphate isomerase (TPI), 13.5 nM glyceraldehyde-3-phosphate dehydrogenase (GPD), and 3.75 nM phosphoglycerate kinase (PGK).

FIG. 7B presents representative data where the seven-enzymes were combined with either 525 QDs, NPLs, or select mixed ratios of both while maintaining a constant total NP concentration of 100 nM. The higher concentration of NPs are utilized here as initial testing showed that the QDs and NPLs tolerated the seven-enzyme system at these concentrations without precipitating. The concentrations and ratios of the enzymes and the NPs utilized in this assay were drawn directly from the previous study (Breger et al., Nature Communications 14 (1), 1757, 2023, incorporated herein by reference). Although using the NPLs alone as the scaffolding material performs quite well, use of 50 nM:50 nM and 25 nM:75 nM ratios of QD:NPL improve the rate of coupled flux beyond that of the NPL alone converting 10-20% more NADH in the same time period. This may seem rather modest, but it is important to appreciate that this improvement was obtained from just a first test assay without any of the extensive parametric testing that was done with the two-enzyme system above. More importantly, this improvement also corresponds to a 4-5 fold more NADH conversion than the QDs are capable of by themselves when used as the sole scaffolding material at the same concentration. Another aspect to note is that total number of (His)₆ tags present is estimated to be ˜95 nM which is on par with the total number of NP present. Enzymes in the seven-enzyme cascade are a mix of monomers, dimers, and tetramers suggesting the total NP concentration should be in slight excess to the total concentration of (His)₆ tags so as to achieve nanocluster formation when further considering NP size and shape.

The seven-enzyme cascade represents a more complex test of the mixed nanoparticle system's capabilities. Each enzyme plays a specific role in the pathway: (i) Glucokinase (GlK) initiates the cascade by phosphorylating glucose using ATP; (ii) Phosphoglucose isomerase (PGI) converts glucose-6-phosphate to fructose-6-phosphate; (iii) Phosphofructokinase (PFK) catalyzes the ATP-dependent phosphorylation to fructose-1,6-bisphosphate; (iv) Aldolase (ALD) cleaves the substrate into dihydroxyacetone phosphate and glyceraldehyde-3-phosphate; (v) Triose phosphate isomerase (TPI) interconverts these three-carbon intermediates; (vi) Glyceraldehyde-3-phosphate dehydrogenase (GAPDH) generates the first high-energy intermediate while reducing NAD+; and (vii) Phosphoglycerate kinase (PGK) couples phosphoryl transfer to ATP synthesis

The sequential nature of this cascade requires careful coordination of reaction rates. Each enzyme must efficiently pass its product to the next enzyme while maintaining overall pathway flux.

The scalability of mixed nanoparticle-enzyme systems requires careful consideration of several factors. The enhanced efficiency of these systems means that smaller reaction volumes can achieve equivalent or superior product yields compared to traditional approaches. However, certain practical considerations become important at larger scales.

The order of component addition becomes particularly critical at larger scales. The optimal assembly protocol involves first combining the nanoparticles in the desired ratio, then adding enzymes sequentially beginning with the final enzyme in the cascade and working backward to the first enzyme. This approach ensures proper cluster formation and optimal enzyme orientation for channeling.

Additionally, the colloidal stability of larger-scale preparations can be maintained by careful control of buffer conditions and total nanoparticle concentration. The mixed QD-NPL systems show superior stability compared to single-particle systems, particularly when handling larger reaction volumes. This enhanced stability stems from the complementary nature of the different nanoparticle geometries, which help prevent excessive aggregation while maintaining desired cluster formation.

Scale-up considerations reveal several critical parameters that become increasingly important at larger volumes. The order and rate of component addition significantly impacts cluster formation, with slower addition rates (approximately 100 μL/min) promoting more uniform assembly. Mixing parameters must be carefully controlled, with gentle orbital mixing preferred over more vigorous stirring methods. Temperature gradients within larger reaction volumes can lead to inhomogeneous assembly, necessitating careful temperature control throughout the entire solution volume. The total protein concentration must be maintained below critical thresholds (typically 500 nM) to prevent unwanted aggregation during scale-up.

Larger scale preparations require modifications to the mixing protocol. Initial mixing is performed using a slow addition rate of approximately 100 μL/min through a syringe pump while maintaining gentle orbital shaking at 100 rpm. The reaction vessel is maintained at a constant temperature using a circulating water bath to prevent temperature gradients. For volumes above 10 mL, the solution is mixed in discrete stages with equilibration periods between additions to ensure uniform cluster formation.

The seven-enzyme cascade system demonstrates the broader applicability of mixed nanoparticle assemblies beyond simple two-enzyme systems. When combining the seven-enzyme cascade with mixed QD-NPL assemblies, several key parameters required optimization:
(1) Total nanoparticle concentration was increased to 100 nM based on stability testing showing that both QDs and NPLs remained colloidally stable at this concentration with the seven-enzyme system.
(2) The ratio of QDs to NPLs was systematically varied while maintaining total nanoparticle concentration, with 50:50 and 25:75 QD:NPL ratios showing optimal performance.
(3) Enzyme concentrations were carefully balanced considering their relative activities: 2.75 nM GlK, 0.5 nM PGI, 4.5 nM PFK, 6 nM ALD, 0.5 nM TPI, 13.5 nM GAPDH, and 3.75 nM PGK.
(4) Buffer conditions were optimized to: 30 mM MgCl2, 15 mM ATP, 15 mM ADP, 20 mM glucose, 8 mM dibasic/monobasic phosphate, 2.25 mM NAD+, and 250 mM HEPES (pH 8).

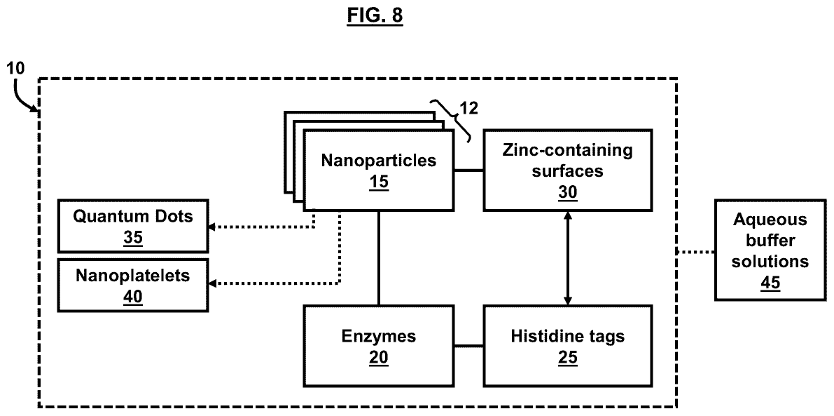

As shown in the block diagram of FIG. 8, with reference to FIGS. 1A through 7C, an embodiment herein provides an enzymatic cluster composition 10 comprising a cluster 12 of nanoparticles 15 having a plurality of enzymes 20 bound thereto via metal affinity coordination between histidine tags 25 on the enzymes 20 and zinc-containing surfaces 30 of the nanoparticles 15. The plurality of enzymes 20 are configured as an enzymatic cascade. The cluster 12 of nanoparticles 15 comprises both quantum dots 35 and nanoplatelets 40. The quantum dots 35 may comprise CdSe/CdS/ZnS core/shell/shell quantum dots. The nanoplatelets 40 may comprise CdSe/ZnS core/shell nanoplatelets.

The enzymatic cluster composition 10 represents a sophisticated nanostructured system for enhancing multi-enzyme reactions through controlled spatial organization. The composition centers around a cluster 12 of nanoparticles 15 that serves as a scaffold to organize and optimize the positioning of multiple enzymes. Both quantum dots 35 and nanoplatelets 40 are incorporated to take advantage of their complementary geometries—the spherical quantum dots provide discrete attachment points while the planar nanoplatelets offer extended surfaces for enzyme binding.

The enzymes 20 are attached to these nanoparticle scaffolds through a specific molecular recognition mechanism involving histidine tags 25 and zinc-containing surfaces 30. Each enzyme 20 displays multiple histidine tags 25 that can coordinate with zinc atoms present on the nanoparticle surfaces 30. This metal coordination forms strong yet dynamic bonds that allow the enzymes to maintain their native conformations and catalytic activity while being held in close proximity to each other.

The composition 10 achieves enhanced catalytic efficiency through several synergistic effects: 1) The mixed nanoparticle geometries create optimized spatial arrangements that place sequential enzymes in an enzymatic cascade close enough for efficient substrate channeling; 2) The multiple attachment points provided by both types of nanoparticles help stabilize enzyme quaternary structure; and 3) The overall cluster architecture maintains colloidal stability while achieving high local enzyme concentrations necessary for improved reaction rates.

The quantum dots 35 may specifically comprise CdSe/CdS/ZnS core/shell/shell structures that provide both the zinc-containing surface for enzyme attachment and useful optical properties for monitoring cluster formation. Similarly, the nanoplatelets 40 may use CdSe/ZnS core/shell architectures to combine enzyme attachment capability with complementary geometric features. Together, these nanoparticle scaffolds create a controlled spatial environment that enhances the natural substrate channeling between cascade enzymes beyond what can be achieved with either particle type alone.

In an example, the quantum dots 35 may have an average diameter between about 4 nm and about 17 nm. This size range for the quantum dots 35 may be utilized for optimal performance of the enzymatic clusters. The lower bound of about 4 nm represents the smallest size that can maintain stable attachment of enzymes 20 while still providing sufficient surface area for the zinc-binding sites. The upper bound of about 17 nm is selected to ensure the quantum dots 35 do not become so large that they disrupt the delicate balance of enzyme proximity and cluster stability. Within this size range, the quantum dots 35 are sufficiently large enough to accommodate multiple enzyme attachments but small enough to fit between nanoplatelets 40 and fill interstitial spaces in the cluster architecture. This size range also corresponds to quantum dots 35 that emit across a useful range of visible wavelengths (525-641 nm), allowing their incorporation into clusters to be monitored spectroscopically. Experimental data with 525 nm emitting (˜4.3 nm), 625 nm emitting (˜9.4 nm), and 641 nm emitting (˜16.9 nm) quantum dots demonstrated that while all sizes within this range could form functional clusters, the smaller quantum dots generally provided better performance by allowing tighter packing and more optimal enzyme arrangements.

In an example, the nanoplatelets 40 may have lateral dimensions between about 15 nm and about 20 nm. The specified lateral dimensions of the nanoplatelets may be utilized to provide ideal surface areas for enzyme attachment and cluster formation. The lateral range of about 15-20 nm may be selected based on several factors: 1) These dimensions are sufficiently large enough to accommodate multiple enzymes 20 across their surface, with each enzyme 20 having a size of approximately 10-15 nm in its tetrameric form; 2) The relatively flat surface allows enzymes 20 to bind in orientations that facilitate substrate channeling between adjacent enzymes 20; and 3) These dimensions create platelets that are small enough to maintain colloidal stability while being large enough to serve as organizational centers for cluster assembly. Experimental studies with CdSe/ZnS nanoplatelets having average dimensions of 19.2×17.3×2.6 nm showed that these lateral dimensions provided an optimal balance between enzyme loading capacity, structural stability, and catalytic enhancement. The quasi-two-dimensional nature of nanoplatelets 40 in this size range also complements the spherical quantum dots by providing extended surfaces that help control overall cluster architecture while preventing excessive aggregation that could occur with larger dimensions.

In an example, the enzymes 20 may be multimeric and display multiple histidine tags 25 that crosslink the nanoparticles 15 into clusters 12. The multimeric nature of the enzymes 20 and their display of multiple histidine tags 25 is fundamental to the formation and function of the nanoparticle-enzyme clusters. In this system, enzymes like pyruvate kinase PykA and lactate dehydrogenase (LDH) exist as homotetramers—structures composed of four identical subunits. Each subunit contains a histidine tag 25, meaning a single enzyme molecule presents four distinct attachment points for binding to nanoparticle surfaces 30. This multiplicity of binding sites enables individual enzyme molecules to simultaneously attach to multiple nanoparticles 15, effectively creating crosslinks between particles. When quantum dots 35 and nanoplatelets 40 are mixed with these multimeric enzymes 20, the multiple attachment points drive the formation of three-dimensional networks where enzymes 20 bridge between different nanoparticles 15. The resulting clusters are stabilized by numerous coordinated bonds between the histidine tags 25 and zinc-containing nanoparticle surfaces 30. This crosslinked architecture is helpful for several reasons: 1) It creates stable assemblies that maintain their structure while remaining colloidally suspended; 2) It positions multiple enzymes 20 in close proximity to enable substrate channeling; and 3) The multiple attachment points help stabilize the quaternary structure of the enzymes 20, often leading to enhanced catalytic activity compared to the free enzymes 20 in solution.

In an example, the plurality of enzymes 20 may comprise pyruvate kinase PykA and lactate dehydrogenase (LDH). The combination of pyruvate kinase PykA and LDH represents a model two-enzyme cascade system that demonstrates aspects of enhanced substrate channeling in mixed nanoparticle clusters 12. These enzymes work sequentially, with PykA first converting phosphoenolpyruvate (PEP) to pyruvate while generating ATP from ADP, followed by LDH converting the pyruvate to lactate while oxidizing NADH to NAD+. When assembled into nanoparticle clusters, these enzymes benefit from their structural characteristics: both exist as homotetramers (PykA˜220 kDa, LDH˜160 kDa) with each monomer displaying a histidine tag, enabling multiple attachment points to the nanoparticle surfaces. The efficiency of this cascade is particularly sensitive to enzyme proximity because the pyruvate intermediate must be efficiently transferred from PykA to LDH before it can diffuse away into bulk solution. In the mixed quantum dot-nanoplatelet clusters, PykA and LDH can be arranged in optimal spatial configurations that facilitate this transfer, leading to demonstrable improvements in reaction rates. The activity of this cascade can be readily monitored through spectroscopic measurements of NADH consumption, making it an excellent system for quantifying the enhancement effects of the mixed nanoparticle clusters compared to single-particle systems or free enzymes in solution.

In an example, the plurality of enzymes 20 may comprise glucokinase, phosphoglucose isomerase, phosphofructokinase, aldolase, triose phosphate isomerase, glyceraldehyde 3-phosphate dehydrogenase, and phosphoglycerate kinase. This seven-enzyme cascade system represents a complex pathway derived from cellular glucose metabolism and demonstrates the scalability of mixed nanoparticle scaffolding to more sophisticated multi-enzyme processes. Each enzyme plays a specific sequential role: glucokinase initiates the cascade by phosphorylating glucose using ATP; phosphoglucose isomerase converts glucose-6-phosphate to fructose-6-phosphate; phosphofructokinase catalyzes a second ATP-dependent phosphorylation; aldolase cleaves the resulting fructose-1,6-bisphosphate into two three-carbon units; triose phosphate isomerase interconverts these intermediates; glyceraldehyde 3-phosphate dehydrogenase generates the first high-energy intermediate while reducing NAD+; and finally, phosphoglycerate kinase couples phosphoryl transfer to ATP synthesis. The successful assembly and enhanced performance of this complex cascade in mixed quantum dot-nanoplatelet clusters is particularly noteworthy because: 1) The enzymes vary in size and quaternary structure (ranging from monomers to tetramers); 2) Each step must be precisely coordinated to prevent bottlenecks or accumulation of intermediates; and 3) The overall pathway involves multiple cofactors (ATP, NAD+) that must remain accessible. The ability of the mixed nanoparticle system to accommodate these diverse requirements while maintaining enhanced activity (10-20% improvement over single-particle systems) demonstrates its broad applicability to complex enzymatic processes.

In an example, the composition 10 may maintain colloidal stability in aqueous buffer solutions 45. The maintenance of colloidal stability in aqueous buffer solutions 45 is helpful for both the formation and function of these enzymatic clusters 12. This stability means the nanoparticle-enzyme assemblies remain uniformly suspended in the buffer without settling, aggregating, or precipitating—a property essential for consistent catalytic activity and practical applications. Several design features contribute to this stability: 1) The surface functionalization of both quantum dots 35 and nanoplatelets 40 with zwitterionic dihydrolipoic acid derivative ligands (CL4) provides electrostatic and steric stabilization; 2) The mixed geometries of spherical quantum dots 35 and planar nanoplatelets 40 help prevent excessive packing that could lead to precipitation; 3) The controlled size of the resulting clusters (typically 50-200 nm) keeps them small enough to remain suspended by Brownian motion; and 4) The buffer conditions (pH, ionic strength, temperature) are optimized to maintain stability while supporting enzyme activity. This colloidal stability enables long-term storage, consistent performance in flow reactors, and compatibility with various bioprocessing applications. Furthermore, the ability to maintain stability in physiologically relevant buffers makes these systems potentially valuable for biotechnology applications requiring sustained activity under biological conditions.

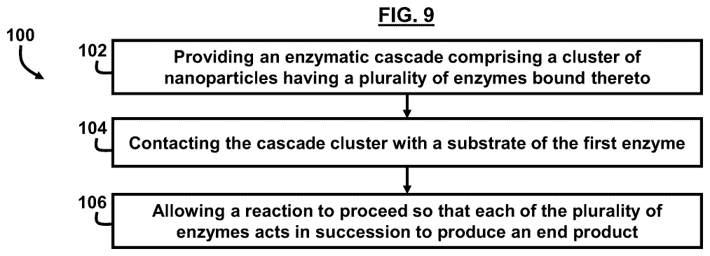

As shown in the flowchart of FIG. 9, with reference to FIGS. 1A through 8, another embodiment provides a method 100 of conducting a cascade reaction, the method 100 comprising providing (102) an enzymatic cascade comprising a cluster 12 of nanoparticles 15 having a plurality of enzymes 20 bound thereto, the plurality of enzymes 20 configured as an enzymatic cascade, and the product of a first enzyme is a substrate of a second enzyme; contacting (104) the cascade cluster with a substrate of the first enzyme; and allowing (106) a reaction to proceed so that each of the plurality of enzymes 20 acts in succession to produce an end product. The cluster 12 of nanoparticles 15 comprises both quantum dots 35 and nanoplatelets 40. The plurality of enzymes 20 are bound to the nanoparticles 15 via metal affinity coordination between histidine tags 25 on the enzymes 20 and zinc-containing surfaces 30 of the nanoparticles 15.

The method 100 provides a systematic approach to conducting cascade reactions using mixed nanoparticle-enzyme clusters 12 that fundamentally differs from traditional solution-phase enzyme catalysis. The first step (102) involves preparation of the cluster assemblies, where quantum dots 35 and nanoplatelets 40 are combined in optimized ratios (typically between 1:5 to 2:1) before sequential addition of histidine-tagged enzymes 20. In an example, the plurality of enzymes 20 may comprise pyruvate kinase PykA and LDH. The metal affinity coordination between histidine tags 25 and zinc-containing nanoparticle surfaces 30 drives spontaneous assembly of stable clusters 12 with controlled enzyme positioning. The second step (104) of contacting these preformed clusters 12 with the initial substrate represents a unique transition point—the substrate must be introduced under conditions that maintain cluster stability while enabling efficient enzyme access. The third step (106) of allowing the reaction to proceed relies on the enhanced substrate channeling created by the optimized spatial arrangement of enzymes 20 within the clusters 12. Each enzyme 20 acts sequentially, with the product of one enzyme being efficiently transferred to the next enzyme in the cascade before it can diffuse into bulk solution. This proximity effect, enabled by the mixed nanoparticle architecture, typically results in reaction rates 2-10 times faster than equivalent reactions with free enzymes or single-particle systems. The success of the method 100 may depend on maintaining appropriate buffer conditions (pH 7.5-8.5, 50-200 mM ionic strength) and temperature (20-25° C.) throughout the reaction to preserve both cluster stability and enzyme activity.

In an example, the quantum dots 35 may comprise CdSe/CdS/ZnS core/shell/shell quantum dots. The specific core/shell/shell architecture of these quantum dots 35 is engineered to optimize both their function as enzyme scaffolds and their stability in aqueous environments. The CdSe core determines the fundamental optical properties of the quantum dots 35, allowing them to serve as spectroscopic markers for monitoring cluster assembly and reaction progress. The intermediate CdS shell helps improve quantum yield and photostability while also acting as a buffer between the core and outer shell. The outer ZnS shell serves multiple critical functions: 1) It provides the zinc-containing surface 30 necessary for metal affinity coordination with the histidine tags 25 of the enzymes 20; 2) It protects the inner core/shell structure from degradation in aqueous buffers; and 3) Its surface chemistry is compatible with the attachment of stabilizing ligands like CL4. The thickness of each shell layer is carefully controlled during synthesis to achieve the desired total particle diameter (ranging from ˜4 nm to ˜17 nm) while maintaining uniform surface properties. This three-layer architecture has proven particularly effective because it combines the required surface chemistry for enzyme attachment with excellent stability and well-defined optical properties that facilitate characterization of the resulting enzyme-nanoparticle clusters 12.

In an example, the nanoplatelets 40 may comprise CdSe/ZnS core/shell nanoplatelets. The core/shell structure of the nanoplatelets 40 combines the beneficial properties of both materials in a two-dimensional geometry. The CdSe core, with precisely controlled thickness of four monolayers, determines the fundamental optical properties of the nanoplatelets 40 and provides the foundation for their unique shape. The ZnS shell serves multiple functions: it provides the zinc-containing surface 30 required for enzyme attachment via histidine coordination, protects the CdSe core from degradation, and enables surface modification with stabilizing ligands. Unlike spherical quantum dots 35, these nanoplatelets 40 have highly anisotropic dimensions (approximately 19.2×17.3×2.6 nm) that create extended flat surfaces ideal for enzyme attachment while maintaining minimal thickness to optimize packing in the resulting clusters.

In an example, the quantum dots 35 and nanoplatelets 40 may be present in a molar ratio of between 1:5 to 2:1. This molar ratio range has been experimentally determined to provide optimal performance in the enzyme cascade systems. The lower bound of 1:5 (QD:NPL) ensures sufficient nanoplatelet surface area for enzyme 20 attachment while still providing enough quantum dots 35 to create effective crosslinking points. The upper bound of 2:1 represents the maximum quantum dot concentration that can be incorporated while maintaining stable cluster formation without excessive aggregation. Within this range, the ratio can be tuned to optimize performance for specific enzyme systems—for example, the two-enzyme PykA/LDH cascade showed best results at ratios around 2:3, while the seven-enzyme system performed optimally at ratios between 1:3 and 1:1.

In an example, the quantum dots 35 may emit at 525 nm. The 525 nm emission wavelength represents an exemplary choice for the quantum dot scaffolds. This wavelength corresponds to quantum dots 35 of approximately 4.3 nm diameter, which provides an ideal size for enzyme attachment while maintaining high surface area to volume ratio. The green emission at 525 nm is easily distinguished from the orange emission of the nanoplatelets 40 (˜585 nm), allowing simultaneous monitoring of both nanoparticle components during cluster assembly and reaction progress. These smaller quantum dots 35 demonstrated superior performance in enzyme cascades compared to larger dots emitting at longer wavelengths (625 nm or 641 nm), likely due to their ability to pack more efficiently in the interstitial spaces between nanoplatelets 40.

In an example, the quantum dots 35 and nanoplatelets 40 may be surface functionalized with zwitterionic dihydrolipoic acid derivative ligands for colloidal stability. The surface functionalization with zwitterionic dihydrolipoic acid derivative ligands (CL4) is helpful for successful cluster formation and stability. These ligands serve multiple functions. For example, they replace the hydrophobic ligands used in nanoparticle synthesis with hydrophilic groups that enable water solubility. Their zwitterionic nature provides electrostatic stabilization while minimizing non-specific interactions that could interfere with enzyme attachment. The dihydrolipoic acid anchor group binds strongly to the ZnS surface without displacing the zinc sites needed for enzyme coordination. The ligand design maintains a thin surface coating that keeps enzymes close to the nanoparticle surface 30 for optimal channeling effects. The CL4 ligands are particularly effective because they provide these functions while being compatible with both the spherical geometry of quantum dots 35 and the planar surfaces of nanoplatelets 40.

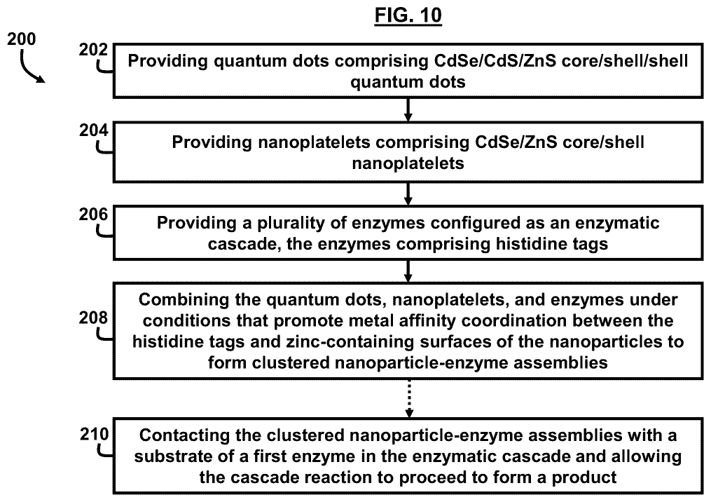

As shown in the flowchart of FIG. 10, with reference to FIGS. 1A through 9, another embodiment provides a method 200 of enhancing enzymatic cascade reactions, the method 200 comprising providing (202) quantum dots 35 comprising CdSe/CdS/ZnS core/shell/shell quantum dots; providing (204) nanoplatelets 40 comprising CdSe/ZnS core/shell nanoplatelets; providing (206) a plurality of enzymes 20 configured as an enzymatic cascade, the enzymes 20 comprising histidine tags 25; and combining (208) the quantum dots 35, nanoplatelets 40, and enzymes 20 under conditions that promote metal affinity coordination between the histidine tags 25 and zinc-containing surfaces 30 of the nanoparticles 15 to form clustered nanoparticle-enzyme assemblies.

The method 200 outlines a systematic approach for enhancing enzymatic cascade reactions through the controlled assembly of mixed nanoparticle-enzyme clusters 12. The method 200 begins with the preparation of two distinct types of nanoparticle scaffolds: quantum dots 35 (step 202) with their three-layer core/shell/shell architecture that provides both structural stability and surface chemistry for enzyme attachment, and nanoplatelets 40 (step 204) that offer complementary planar surfaces for enzyme organization. The next step (206) involves preparing the histidine-tagged enzymes 20 under conditions that maintain their native quaternary structure and catalytic activity. Thereafter, the combining step (208) is performed in a carefully controlled manner to achieve optimal cluster formation. The combination typically proceeds by first mixing the quantum dots 35 and nanoplatelets 40 in the desired ratio (usually between 1:5 and 2:1) in buffer at pH 8.0, followed by sequential addition of enzymes 20 starting with those later in the cascade. The metal affinity coordination between histidine tags 25 and zinc-containing surfaces 30 drives spontaneous assembly under these conditions, but the process includes careful control of temperature (4-25° C.), ionic strength (50-200 mM), and total protein concentration (typically below 500 nM) to achieve stable clusters with enhanced catalytic activity. This method 200 has demonstrated consistent success in creating functional enzyme cascades that show 2-10 fold enhancement in reaction rates compared to traditional solution-phase approaches.

In an example, the quantum dots 35 and nanoplatelets 40 may be provided in a molar ratio of between 1:5 to 2:1. In an example, the plurality of enzymes 20 may comprise pyruvate kinase PykA and LDH. In an example, the plurality of enzymes 20 may comprise glucokinase, phosphoglucose isomerase, phosphofructokinase, aldolase, triose phosphate isomerase, glyceraldehyde 3-phosphate dehydrogenase, and phosphoglycerate kinase.

In an example, the clustered nanoparticle-enzyme assemblies may stabilize a quaternary structure of at least one enzyme in the enzymatic cascade. The stabilization of the enzyme quaternary structure by the clustered nanoparticle assemblies represents a unique aspect of the embodiments herein beyond simple substrate channeling effects. Many enzymes, such as LDH which exists as a homotetramer of ˜160 kDa, require specific quaternary arrangements of their subunits for optimal activity. The mixed nanoparticle clusters 12 help maintain these critical structures through multiple mechanisms. For example, the multiple histidine tags 25 on each enzyme 20 create several attachment points to the nanoparticle surfaces 30, effectively cross-bracing the quaternary structure. The complementary geometries of quantum dots 35 and nanoplatelets 40 provide diverse surface environments that can accommodate and stabilize different protein conformations. The local environment around the nanoparticle surfaces 30, including controlled pH and ionic strength, helps maintain proper subunit interactions. The physical constraint of being bound within the cluster 12 protects against subunit dissociation that might occur in free solution.

In an example, the method 200 may further comprise contacting (210) the clustered nanoparticle-enzyme assemblies with a substrate of a first enzyme in the enzymatic cascade and allowing the cascade reaction to proceed to form a product. This allows the full cascade reaction to proceed, with the product of each enzyme in the cascade being efficiently transferred to the next enzyme due to the optimized spatial arrangement and proximity enabled by the mixed nanoparticle scaffold. By allowing the cascade reaction to proceed with the substrate added to the pre-formed clusters, the enhanced catalytic efficiency of the system can be realized, ultimately producing the desired end product. The method 200 starts by preparing the key components—the quantum dots 35, nanoplatelets 40, and enzymes 20 configured in a cascade. These are then combined under conditions that promote the formation of the clustered nanoparticle-enzyme assemblies (steps 202-208). Step 210 involves taking these pre-formed clustered assemblies and contacting them with the substrate of the first enzyme in the cascade. This allows the complete cascade reaction to take place, with each enzyme in the sequence acting on its substrate and passing the product along to the next enzyme. One aspect of this approach is that the optimized spatial arrangement of the enzymes 20 within the mixed nanoparticle clusters 12 facilitates efficient substrate channeling between the sequential steps of the cascade. Rather than the intermediate products diffusing away into bulk solution, they are rapidly transferred from one active site to the next. Furthermore, this enhanced substrate channeling, enabled by the mixed quantum dot and nanoplatelet scaffolding, leads to significantly improved reaction rates and product yields compared to free enzymes in solution or single nanoparticle systems. By pre-assembling the clusters first, the cascade reaction can then proceed in a highly efficient manner when the initial substrate is introduced.

### Further Embodiments

The above examples employed certain ratios in combining QDs and NPLs, but these can be varied by one of skill in the art. As can be seen in FIG. 6A, in the two-enzyme system, the best-performing samples had a QD:NPL molar ratio of 2:3 (corresponding to 0.4 nM QD and 0.6 nM NPL) and 1:4 (corresponding to 0.2 nM) whereas, considering the seven-enzyme cascade data of FIG. 7B, the two best-performing samples had a QD:NPL molar ratio of 1:3 and 1:1. Accordingly, QD:NPL molar ratios ranging from at least 1:5 to 2:1 are contemplated.

While specific embodiments using CdSe-based quantum dots and nanoplatelets are described, other semiconductor materials could be employed including but not limited to: ZnS-based materials, InP-based materials, and other II-VI or III-V semiconductor compositions. The principles described herein could also extend to other types of inorganic nanoparticles with appropriate surface chemistry for enzyme attachment.

Direct comparison between mixed nanoparticle systems and other approaches for enzyme cascade optimization reveals several key advantages. Unlike enzyme fusion approaches, which require extensive protein engineering and may not maintain optimal activity of all components, the mixed nanoparticle system allows each enzyme to maintain its native structure while achieving proximity effects. Compared to DNA-based scaffolding approaches, the mixed nanoparticle system provides greater structural stability and simpler assembly protocols.

The performance improvements observed in mixed nanoparticle systems extend beyond simple additive effects of the individual components. The synergistic enhancement observed suggests that the combination of different nanoparticle geometries creates unique organizational structures that are particularly effective for substrate channeling. This synergy is especially evident in the seven-enzyme system, where the complexity of the cascade might be expected to reduce efficiency but instead shows marked improvement in mixed systems.

The economic advantages of mixed nanoparticle systems also merit consideration. The improved catalytic efficiency means that lower enzyme concentrations can achieve equivalent or superior product formation compared to traditional approaches. The stability of these systems reduces the need for frequent enzyme replacement, while the simplified assembly process minimizes preparation complexity and cost.

### Advantages

Mixed nanoparticle systems (QDs combined with NPLs) demonstrated a >10× improvement in k_{flux} is observed relative to the free enzymes which is 2× greater than that of the individual nanoparticles. These results suggest that the ability to control and tune the geometric packing motif enables formation of an immobilized enzyme cluster which has been structurally optimized for product formation via intermediary channeling in vitro. The ability to develop mixed nanoparticle scaffolds that increase the overall rate of the reaction offers the following advantages, benefits, and characteristics:
(1) The ability to design mixed nanoparticle scaffolds which improve the structural environment to further enhance enzymatic channeling beyond what can be achieved with a single type of nanoparticle material.
(2) The ability to control and tune the underlying geometric packing motif enables formation of an immobilized enzyme cluster which has been structurally optimized for product formation via intermediary channeling in vitro.
(3) The ability to increase the efficiency of a biocatalytic reaction via mixed nanoparticle systems.
(4) The ability to increase the rate of overall kinetic flux via mixed nanoparticle systems.
(5) The ability to tune the material used in a channeling mechanism to a given enzyme and/or reaction step that is most suitable for accessing substrate channeling.
(6) The ability to utilize the NP aggregated portion of the system to stabilize the enzymes for long-term use.
(7) The ability to exploit these processes for other multienzyme systems.
(8) The ability of the mixed nanoparticle-enzyme cluster system to have extended colloidal stability versus assemblies with just one type of nanoparticle.
(9) The ability to mix and match multiple nanoparticle sizes/shapes beyond just two to further optimize the systems.
(10) The ability to change and match both nanoparticle sizes/shapes and ratios along with enzyme ratios to further optimize the systems.
(11) Enhanced stability of enzyme quaternary structure in the mixed nanoparticle assemblies.
(12) Improved product yields with reduced substrate concentrations.
(13) Greater synthetic versatility compared to cell-based systems through ability to work with non-natural substrates.
(14) Ability to fine-tune reaction kinetics through systematic optimization of nanoparticle ratios.
(15) Capability to stabilize and enhance activity of enzymes with significantly different catalytic rates within the same cascade.

The practical implementation of mixed nanoparticle-enzyme systems requires attention to several key operational parameters. Solution preparation follows a specific sequence to ensure optimal assembly. The nanoparticles are first combined in buffer at their designated ratio and allowed to equilibrate. Enzymes are then added sequentially, beginning with the final enzyme in the cascade pathway. This ordered addition helps establish proper spatial arrangements for efficient substrate channeling.

Temperature control during both assembly and reaction proves critical for optimal performance. Assembly proceeds optimally at 4° C., while reactions typically perform best at room temperature (20-25° C.). The system maintains stability across this temperature range without significant loss of activity. pH control requires careful monitoring, with optimal performance typically observed between pH 7.5-8.5.

Long-term stability studies were conducted under various storage conditions. Samples stored at 4° C. in standard buffer maintained >90% of initial activity for at least 4 weeks when supplemented with 20% glycerol. Multiple freeze-thaw cycles were found to be detrimental to activity, with approximately 15-20% loss per cycle. Room temperature storage resulted in gradual activity decline, with approximately 50% activity remaining after one week. The addition of stabilizing agents such as bovine serum albumin (0.1 mg/mL) provided modest improvements in room temperature stability.

Activity measurements during storage stability studies were performed weekly using standardized assay conditions. The loss of activity followed first-order kinetics with a half-life of approximately 30 days at 4° C. when stored with 20% glycerol. Room temperature samples showed accelerated activity loss with degradation rates 3-4 times faster than refrigerated samples. The addition of stabilizing agents was screened using a panel of common protein stabilizers including trehalose, polyethylene glycol, and various amino acids.

Storage of pre-assembled systems remains viable for extended periods when maintained at 4° C. in appropriate buffer conditions. The addition of glycerol to 20-30% can further enhance storage stability without compromising activity upon subsequent use.

This mixed nanoparticle approach to enzyme cascade optimization suggests several promising directions for future development. The principles established here could be extended to other nanoparticle types including metal nanoparticles, carbon-based materials, and other semiconductor nanostructures. The ability to combine three or more different types of nanoparticles may provide even greater control over cluster architecture and enzyme positioning.

The demonstrated enhancement of both simple two-enzyme and more complex seven-enzyme cascades suggests broad applicability across different enzyme systems. This approach could be particularly valuable for cascades involving challenging intermediate products or significant differences in optimal conditions between component enzymes.

All literature and documents mentioned herein are hereby incorporated by reference for the purpose of disclosing and describing the particular materials and methodologies for which the document was cited.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Terminology used herein should not be construed as being “means-plus-function” language unless the term “means” is expressly used in association therewith. Those skilled in the art will recognize that the embodiments herein can be practiced with modification within the spirit and scope of the appended claims.

What is claimed is:

1. A method of conducting a cascade reaction, the method comprising:
providing an enzymatic cascade comprising a cluster of nanoparticles having a plurality of enzymes bound thereto, the plurality of enzymes configured as an enzymatic cascade, wherein the product of a first enzyme is a substrate of a second enzyme;
contacting the cascade cluster with a substrate of the first enzyme; and
allowing a reaction to proceed so that each of the plurality of enzymes acts in succession to produce an end product,
wherein the cluster of nanoparticles comprises both quantum dots and nanoplatelets, and
wherein the plurality of enzymes are bound to the nanoparticles via metal affinity coordination between histidine tags on the enzymes and zinc-containing surfaces of the nanoparticles.

2. The method of claim 1, wherein the quantum dots comprise CdSe/CdS/ZnS core/shell/shell quantum dots.

3. The method of claim 1, wherein the nanoplatelets comprise CdSe/ZnS core/shell nanoplatelets.

4. The method of claim 1, wherein the quantum dots and nanoplatelets are present in a molar ratio of between 1:5 to 2:1.

5. The method of claim 1, wherein the quantum dots emit at 525 nm.

6. The method of claim 1, wherein the quantum dots and nanoplatelets are surface functionalized with zwitterionic dihydrolipoic acid derivative ligands for colloidal stability.

7. The method of claim 1, wherein the plurality of enzymes comprises pyruvate kinase PykA and lactate dehydrogenase.

8. An enzymatic cluster composition comprising:
a cluster of nanoparticles having a plurality of enzymes bound thereto via metal affinity coordination between histidine tags on the enzymes and zinc-containing surfaces of the nanoparticles,
wherein the plurality of enzymes are configured as an enzymatic cascade,
wherein the cluster of nanoparticles comprises both quantum dots and nanoplatelets,
wherein the quantum dots comprise CdSe/CdS/ZnS core/shell/shell quantum dots, and
wherein the nanoplatelets comprise CdSe/ZnS core/shell nanoplatelets.

9. The enzymatic cluster composition of claim 8, wherein the quantum dots have an average diameter between about 4 nm and about 17 nm.

10. The enzymatic cluster composition of claim 8, wherein the nanoplatelets have lateral dimensions between about 15 nm and about 20 nm.

11. The enzymatic cluster composition of claim 8, wherein the enzymes are multimeric and display multiple histidine tags that crosslink the nanoparticles into clusters.

12. The enzymatic cluster composition of claim 8, wherein the plurality of enzymes comprises pyruvate kinase and lactate dehydrogenase.

13. The enzymatic cluster composition of claim 8, wherein the plurality of enzymes comprises glucokinase, phosphoglucose isomerase, phosphofructokinase, aldolase, triose phosphate isomerase, glyceraldehyde 3-phosphate dehydrogenase, and phosphoglycerate kinase.

14. The enzymatic cluster composition of claim 8, wherein the composition maintains colloidal stability in aqueous buffer solutions.

15. A method of enhancing enzymatic cascade reactions, the method comprising:
providing quantum dots comprising CdSe/CdS/ZnS core/shell/shell quantum dots;
providing nanoplatelets comprising CdSe/ZnS core/shell nanoplatelets;
providing a plurality of enzymes configured as an enzymatic cascade, wherein the enzymes comprise histidine tags; and
combining the quantum dots, nanoplatelets, and enzymes under conditions that promote metal affinity coordination between the histidine tags and zinc-containing surfaces of the nanoparticles to form clustered nanoparticle-enzyme assemblies.

16. The method of claim 15, wherein the quantum dots and nanoplatelets are provided in a molar ratio of between 1:5 to 2:1.

17. The method of claim 15, wherein the plurality of enzymes comprises pyruvate kinase and lactate dehydrogenase.

18. The method of claim 15, wherein the plurality of enzymes comprises glucokinase, phosphoglucose isomerase, phosphofructokinase, aldolase, triose phosphate isomerase, glyceraldehyde 3-phosphate dehydrogenase, and phosphoglycerate kinase.

19. The method of claim 15, wherein the clustered nanoparticle-enzyme assemblies stabilize a quaternary structure of at least one enzyme in the enzymatic cascade.

20. The method of claim 15, further comprising contacting the clustered nanoparticle-enzyme assemblies with a substrate of a first enzyme in the enzymatic cascade and allowing the cascade reaction to proceed to form a product.


SEQUENCE LISTING (2 sequences)

SEQ ID NO: 1 - protein, 500 aa, Escherichia coli
MGSSHHHHHHSSGLVPRGSHMSRRLRRTKIVTTLGPATDRDNNLEKVIAAGANVVRMNFSHGSPEDHKMRADKVREIAAKLGRHVAILGDLQGPKIRVSTFKEGKVFLNIGDKFLLDANLGKGEGDKEKVGIDYKGLPADVVPGDILLLDDGRVQLKVLEVQGMKVFTEVTVGGPLSNNKGINKLGGGLSAEALTEKDKADIKTAALIGVDYLAVSFPRCGEDLNYARRLARDAGCDAKIVAKVERAEAVCSQDAMDDIILASDVVMVARGDLGVEIGDPELVGIQKALIRRARQLNRAVITATQMMESMITNPMPTRAEVMDVANAVLDGTDAVMLSAETAAGQYPSETVAAMARVCLGAEKIPSINVSKHRLDVQFDNVEEAIAMSAMYAANHLKGVTAIITMTESGRTALMTSRISSGLPIFAMSRHERTLNLTALYRGVTPVHFDSANDGVAAASEAVNLLRDKGYLMSGDLVIVTQGDVMSTVGSTNTTRILTVE

SEQ ID NO: 2 - protein, 349 aa, Escherichia coli
MGSSHHHHHHSSGLVPRGSHMKLAVYSTKQYDKKYLQQVNESFGFELEFFDFLLTEKTAKTANGCEAVCIFVNDDGSRPVLEELKKHGVKYIALRCAGFNNVDLDAAKELGLKVVRVPAYDPEAVAEHAIGMMMTLNRRIHRAYQRTRDANFSLEGLTGFTMYGKTAGVIGTGKIGVAMLRILKGFGMRLLAFDPYPSAAALELGVEYVDLPTLFSESDVISLHCPLTPENYHLLNEAAFDQMKNGVMIVNTSRGALIDSQAAIEALKNQKIGSLGMDVYENERDLFFEDKSNDVIQDDVFRRLSACHNVLFTGHQAFLTAEALTSISQTTLQNLSNLEKGETCPNELV